(19) United States
(12) Patent Application Publication
(10) Pub. No.: US 2013/0273547 A1
(43) Pub. Date: Oct. 17, 2013

(54) METHOD TO DETERMINE AND CORRECT BASELINE AND TO CHARACTERIZE PCR AMPLIFICATION KINETICS

(71) Applicant: SAMSUNG TECHWIN CO., LTD., Changwon-city (KR)

(72) Inventor: Jun LI, Beltsville, MD (US)

(73) Assignee: Samsung Techwin Co., Ltd., Changwon-city (KR)

(21) Appl. No.: 13/852,078

(22) Filed: Mar. 28, 2013

Related U.S. Application Data
(60) Provisional application No. 61/624,597, filed on Apr. 16, 2012.

(51) Int. Cl. C12Q 1/68

(52) U.S. Cl. CPC C12Q 1/686; G06F 19/18
USPC 435/6.12; 702/19

(57) ABSTRACT

The disclosed methods correct for high-sloped baseline observed in real-time PCR curves and provides a way of verifying amplification efficiency using a statistical approach. The method teaches the conversion of fluorescent data points to transform the baseline from sloped to horizontal. Then, a 4-parameter logistic model is applied to simulate the PCR kinetics. A cycle number corresponding to a template concentration can then be determined at a inflection point defined by the model

### FIELD

The disclosure describes baseline determination and correction and S-shape curve regression methods for improving the quantification of target nucleic acids by real-time PCR detection.

### BACKGROUND

Real-time PCR technologies have revolutionized molecular diagnostic applications because they permit the monitoring of PCR amplification kinetics, which permits the accurate quantification of target nucleic acids with the requisite level of sensitivity. For example, with fluorescent dual-labeled hybridization probe technologies such as the “CATACLEAVE™ endonuclease assay (described in detail in U.S. Pat. No. 5,763,181; see FIGS. 1 and 2), detection of target nucleic acid sequences is achieved by including a labeled CATACLEAVE™ probe in the amplification reaction together with an RNase H activity. The CATACLEAVE™ probe, which is complementary to a target sequence within the PCR amplification product, has a chimeric structure comprising an RNA sequence and a DNA sequence, and is flanked at its 5′ and 3′ ends by a detectable marker, for example, FRET pair labeled DNA sequences. The proximity of the FRET pair's fluorescent label to the quencher precludes fluorescence of the intact probe. However, annealing of the probe to the PCR product generates an RNA: DNAduplex that can be cleaved by RNase H present in the amplification buffer. Cleavage of the duplexed RNA sequences results in the separation of the fluorescent label from the quencher and a subsequent emission of fluorescence. The concentration of the template nucleic acid can then be deduced from the number of cycles required to achieve a given level of fluorescence.

In the traditional cycle threshold (Ct) method the cycle number is determined based on the point within the exponential phase of the PCR curve where the fluorescence response increases above the baseline level to cross a predetermined fluorescence threshold value. This approach is however subject to errors caused by signal anomalies that distort the baseline fluorescence level and decrease the efficiency and sensitivity of real-time PCR amplification. Alternatively, the cycle number can be determined mathematically. For example, the Second Derivative Maximum method identifies the crossing point (Cp) of a sample as the point where the sample's fluorescence curve turns sharply upward, i.e., Cp corresponding to the maximum of the second derivative of the amplification curve. This method alleviates burdens on the user side to determine a “fluorescence threshold”. However, this approach only works on perfect symmetric S-shaped PCR curve, and can be prone to errors in the presence distorted baseline fluorescence signals.

For the foregoing reasons, there is an unmet need in the art for improved real-time PCR protocols.

### SUMMARY

The disclosed methods apply to real-time PCR curves with a flat or sloped baseline. The approach improves the sensitivity of the real-time PCR reaction and also provides a means of verifying the amplification efficiency. The sloped baseline of a real-time PCR curve is first converted to a curve with a flat baseline having near zero fluorescence. No conversion is necessary if the baseline is “flat”, or zero sloped. A 4-parameter Logistic regression is then applied to simulate the PCR kinetics. A cycle number corresponding to a template concentration can then be determined at an inflection point defined by the model.

In one embodiment, a method of correcting a real-time PCR curve with a sloped baseline is disclosed, comprising:

detecting an optical signal emitted during a real-time PCR amplification of a target nucleic acid;

plotting the intensity of the optical signal as a function of cycle number to obtain a first real-time PCR plot;

distinguishing a baseline phase from an exponential phase;

generating a baseline slope fitting curve of the first plot;

converting the first plot to a second plot with a flat baseline;

transforming the second plot into a third plot having a baseline that is minimized to almost zero fluorescence, and

determining the concentration of the target nucleic acid in the sample.

The baseline phase can be distinguished from the exponential phase individually and uniquely for each sample well and/or each dye being evaluated. For each sample well and/or dye, a first plot is generated by plotting the actual signals generated by a label (e.g., fluorescein) in the sample well as a function of cycle number. In a preferred embodiment, a second plot is generated by plotting a 3-point moving Coefficient of Variance (CV) of the actual signals as a function of cycle number by applying the equation

$${CV}_{i} = 100 \% \times \frac{{σ}_{i}}{{y}_{i}}$$

wherein CVᵢ is coefficient of variance of the amount of fluorescence cycles i−1, i, and i+1, and σᵢ is standard deviation of the amount of fluorescence at cycles i−1, i, and i+1, and [symbol] ᵢ is the amount of fluorescence at cycle i. The end cycle of the baseline phase or the starting cycle of the exponential phase is assigned as one of the followings: 1) a first cycle which shows a CV significantly greater than that of its previous cycle, and 2) a cycle having a CV that is equal to or greater than a specified value, e.g., 0.5. If no cycle satisfies the criteria in 1) and 2), the end cycle of the baseline phase (or the starting cycle of the exponential phase) is assigned as the total PCR cycles. The starting cycle of the baseline phase can be cycle 1, 2, 3 or any specified cycle number. A method for calculating a 3-point CV is shown in Table 1.

TABLE 1
Calculating 3-point CV

| Cycle | 1 | 2 | 3 | 4 |
|---|---|---|---|---|
| Fluorescence | Y₁ | Y₂ | Y₃ | Y₄ |
| signal | | | | |
| 3-point average fluorescence signal | | $\overline{{\overline{Y}}_{2}} = \frac{{Y}_{1} + {Y}_{2} + {Y}_{3}}{3}$ | $\overline{{\overline{Y}}_{3}} = \frac{{Y}_{2} + {Y}_{3} + {Y}_{4}}{3}$ | $\overline{{\overline{Y}}_{4}} = \frac{{Y}_{3} + {Y}_{4} + {Y}_{5}}{3}$ |
| 3-point standard deviation | | ${σ}_{2} = \sqrt{\frac{\begin{matrix}{( {Y}_{1} - \overline{{Y}_{2}} )}^{2} + {( {Y}_{2} - \overline{{Y}_{2}} )}^{2} + \\ {( {Y}_{3} - \overline{{Y}_{2}} )}^{2}\end{matrix}}{2}}$ | ${σ}_{3} = \sqrt{\frac{\begin{matrix}{( {Y}_{2} - \overline{{Y}_{3}} )}^{2} + {( {Y}_{3} - \overline{{Y}_{3}} )}^{2} + \\ {( {Y}_{4} - \overline{{Y}_{3}} )}^{2}\end{matrix}}{2}}$ | ${σ}_{4} = \sqrt{\frac{\begin{matrix}{( {Y}_{3} - \overline{{Y}_{4}} )}^{2} + {( {Y}_{4} - \overline{{Y}_{4}} )}^{2} + \\ {( {Y}_{5} - \overline{{Y}_{4}} )}^{2}\end{matrix}}{2}}$ |
| 3-point CV | | ${CV}_{2} = 100 \% \frac{{σ}_{2}}{{Y}_{2}}$ | ${CV}_{3} = 100 \% \frac{{σ}_{3}}{{Y}_{3}}$ | ${CV}_{4} = 100 \% \frac{{σ}_{4}}{{Y}_{4}}$ |

The baseline slope fitting curve can be a linear regression that is determined by applying the equation

y= [symbol] x+

[symbol]

to each fluorescence data point of the first plot's baseline, wherein [symbol] is the amount of fluorescence at cycle x, [symbol] is slope of the baseline fitting curve, and [symbol] is the value at the intercept of the curve with the abscissa axis. Cycle x can start at cycle 1 and increase incrementally until the beginning of the first real-time PCR plot's exponential phase. Validity or linearity of baseline fitting curve can be determined by Square of the Pearson Coefficient (R²) by applying the equation

$${R}^{2} = \frac{{\sum }_{i = m}^{n} ( {x}_{i} - \overline{x} ) {( {y}_{i} - \overline{y} )}^{2}}{{\sum }_{i = m}^{n} {( {x}_{i} - \overline{x} )}^{2} {\sum }_{i = m}^{n} {( {y}_{i} - \overline{y} )}^{2}}$$

wherein m is the starting cycle of the baseline phase, and n is the end cycle of the baseline phase, [symbol] ᵢ is the amount of fluorescence at cycle xᵢ, y̅ is the average fluorescence of the baseline, and x̅ is the average cycle number of the baseline.

The third plot with a flat baseline is produced by converting each fluorescent data point (x, [symbol] ) of the first real-time PCR plot into a modified fluorescent data point (x′, [symbol] ′) by applying the equation:

$$[ \begin{matrix}{x}^{′} \\ {y}^{′}\end{matrix} ] = [ \begin{matrix}cos θ & - sin θ \\ sin θ & cos θ\end{matrix} ] [ \begin{matrix}x \\ y\end{matrix} ]$$

to each fluorescent data point of the first real-time PCR plot, wherein θ is the angle corresponding to the slope of the baseline fitting curve.

The fourth plot can be generated with fluorescent data points that are corrected to minimize baseline fluorescence, each corrected fluorescent data point, [symbol] ″ᵢ, at cycle xᵢ, being determined by subtracting the average fluorescence value of baseline, y̅′̅z, from each modified fluorescence value ( [symbol] ′ᵢ) by applying the equation: [symbol] ″ᵢ= [symbol] ′ᵢ−

[symbol]

wherein the average value of the baseline, y̅′̅z, is calculated by applying the equation:

$$\overline{{y}^{′}} = \frac{{{\sum }_{i = 1}}^{n} {y}_{i}^{′}}{n} ,$$

wherein [symbol] ′ᵢ is the value of the modified fluorescent data point at cycle xᵢ, and xₙ is the total cycle number included in the baseline calculation.

In another embodiment, the disclosure teaches a method for determining an S-shape curve function that fits a baseline slope corrected real-time PCR curve, comprising:

detecting an optical signal emitted during a real-time PCR amplification of a target nucleic acid;

plotting the intensity of the optical signal as a function of cycle number to obtain a first real-time PCR plot;

transforming the first real-time PCR plot into a baseline slope corrected second plot;

correlating an S-shape curve function from the baseline slope corrected second plot.

The correlation of the S-shape curve function with the baseline slope corrected second plot can include the steps of applying the equation

$$f ( x ) = {y}_{0} + \frac{a}{1 + {( \frac{x}{{x}_{0}} )}^{b}}$$

to each fluorescent data point of the baseline corrected second plot, wherein [symbol] (x) is the value of function computed for a fluorescence data point at cycle x, [symbol] ₀ is the ground fluorescence, a is the difference between the maximal fluorescence acquired in the run and the ground fluorescence, x is the actual cycle number, x₀ is the first derivative maximum of the function, and [symbol] describes the slope of the curve at x₀, and is defined by the equation −4· [symbol] (x₀)·x₀/a.

The rotational transformation of the first real-time PCR plot into a baseline slope corrected plot can include the steps of determine a baseline phase, generating a baseline slope fitting curve of the first real-time PCR plot, rotationally transforming the first real-time PCR plot to a second plot with a flat baseline; and then correcting the flat-baseline plot into another plot having a baseline that is minimized to almost zero fluorescence.

The second plot with a flat baseline can be produced by converting each fluorescent data point (x, [symbol] ) into a modified fluorescent data point (x′, [symbol] ′) by applying the equation

$$[ \begin{matrix}{x}^{′} \\ {y}^{′}\end{matrix} ] = [ \begin{matrix}cos θ & - sin θ \\ sin θ & cos θ\end{matrix} ] [ \begin{matrix}x \\ y\end{matrix} ]$$

to each fluorescent data point of the first real-time PCR plot, wherein θ is the angle corresponding to the slope of the baseline fitting curve.

The third plot can be generated with fluorescent data points that are corrected to minimize baseline fluorescence, each corrected fluorescent data point, [symbol] ″ᵢ, at cycle xᵢ, being determined by subtracting the average fluorescence value of baseline, z, from each modified fluorescence value ( [symbol] ′ᵢ) by applying the equation: [symbol] ″ᵢ= [symbol] ′ᵢ−

[symbol]

wherein the average value of the baseline, y̅′̅z, is calculated by applying the equation

$$\overline{{y}^{′}} = \frac{{\sum }_{i = 1}^{n} {y}_{i}^{′}}{n} ,$$

and wherein [symbol] ′ᵢ is the value of the modified fluorescent data point at cycle xᵢ, and xₙ is the total cycle number included in the baseline calculation.

In another embodiment, the application discloses a method of determining the crossing point cycle, Cᵢₜ, can include:

detecting an optical signal emitted during a real-time PCR amplification of a target nucleic acid;

plotting the intensity of the optical signal as a function of cycle number to obtain a first real-time PCR plot;

distinguishing a baseline phase from an exponential phase;

transforming the first real-time PCR plot into a baseline slope corrected plot having a baseline that is minimized to almost zero fluorescence,

correlating an S-shape curve function from the baseline slope corrected plot; and

determining a Cᵢₜ value at the intersection point between the abscissa axis and tangent of the inflection point of the S-shape curve function;

wherein the Cᵢₜ value is a measurement of the concentration of the nucleic acid in the sample.

The inflection point coordinate can be calculated by applying the equation:

$$inflection point = [ {x}_{0} , {y}_{0} + \frac{a}{2} ]$$

wherein the slope of the tangent line at x₀ is [symbol] ′(x₀), and the Cᵢₜ value is calculated with the equation:

$${C}_{it} = - \frac{{y}_{0} + \frac{a}{2}}{{f}^{′} ( {x}_{0} )} cos θ .$$

The transformation of the first real-time PCR plot into a second plot having a baseline that is minimized to almost zero fluorescence can include the steps of: producing the second plot with a flat baseline by converting each fluorescent data point (x, [symbol] ) of the real-time PCR plot into a modified fluorescent data point (x′, [symbol] ′) by applying the equation:

$$[ \begin{matrix}{x}^{′} \\ {y}^{′}\end{matrix} ] = [ \begin{matrix}cos θ & - sin θ \\ sin θ & cos θ\end{matrix} ] [ \begin{matrix}x \\ y\end{matrix} ] ,$$

wherein θ is the angle corresponding to the slope of the baseline fitting curve; and determining a corrected fluorescent data point, [symbol] ″ᵢ, at cycle xᵢ, from the baseline fitting curve by subtracting the average fluorescence value of the baseline, y̅′̅z, from each modified fluorescence value ( [symbol] ′ᵢ) by applying the equation: [symbol] ″ᵢ= [symbol] ′ᵢ− [symbol] , wherein the average value of the baseline, y̅′̅z, is calculated by applying the equation:

$$\overline{{y}^{′}} = \frac{{\sum }_{i = 1}^{n} {y}_{i}^{′}}{n} ,$$

wherein [symbol] ′ᵢ is the value of the modified fluorescent data point at cycle xᵢ, and xₙ is the total cycle number included in the baseline calculation.

The correlation of the S-shape curve function with the baseline slope corrected plot can include the steps of applying the equation

$$f ( x ) = {y}_{0} + \frac{a}{1 + {( \frac{x}{{x}_{0}} )}^{b}} ,$$

wherein [symbol] (x) is the value of the function computed for a fluorescence data point at cycle x, [symbol] ₀ is the ground fluorescence, a, is the difference between the maximal fluorescence acquired in the run and the ground fluorescence, x is the actual cycle number, x₀ is the first derivative maximum of the function, and [symbol] describes the slope of the curve at x₀, and is defined as −4· [symbol] (x₀)·x₀/a.

The real-time PCR amplification can have the steps of:

providing a sample to be tested for the presence of a target DNA sequence;

providing a pair of forward and reverse amplification primers, wherein the forward amplification primer anneals to the 5′ end of the target nucleic acid sequence and the reverse amplification primer anneals to the 3′ end of the target nucleic acid sequence;

providing a probe comprising a detectable label and DNA and RNA nucleic acid sequences, wherein the probe's RNA nucleic acid sequences are entirely complementary to a selected region of the target DNA and the probe's DNA nucleic acid sequences are substantially complementary to sequences adjacent to the selected region of the target DNA sequence, and

amplifying a PCR fragment between the forward and reverse amplification primers in the presence of the target DNA sequence, an amplifying polymerase and an amplification buffer comprising a thermostable RNase H activity under conditions where the RNA sequences within the probe can form a RNA:DNA heteroduplex with complimentary sequences in the PCR fragment, wherein cleavage of RNA sequences within the RNA:DNA heteroduplex by the RNase H activity results in the emission of the optical signal from the label on the probe.

The detectable label on the probe can be a fluorescent label such as a FRET pair.

The target nucleic acid sequence can be a cDNA of a target RNA sequence.

The previously described embodiments have many advantages, including the ability to correct real-time PCR curves for errors caused by signal anomalies that distort the baseline fluorescence level and decrease the efficiency and sensitivity of real-time PCR amplification.

### BRIEF DESCRIPTION OF THE DRAWINGS

The skilled artisan will understand that the drawings, described below, are for illustration purposes only. The figures are not intended to limit the scope of the teachings in any way.

FIG. 1 is a schematic representation of CataCleave™ probe technology.

FIG. 2 is a schematic representation of real-time CataCleave™ probe detection of PCR amplification products.

FIG. 3 shows 3-point CV plotted vs. cycle number observed in a real-time PCR/CataCleave fluorescence curve in (x, [symbol] ) coordinates.

FIG. 4 depicts a linear regression fitting of a baseline observed in a real-time PCR/CataCleave fluorescence curve in (x, [symbol] ) coordinates. In this example, the baseline fitting starts from cycle 3 and ends at cycle 12.

FIG. 5 depicts a fluorescence curve that is rotated counter-clockwise by θ to “flatten” the baseline in (x′, [symbol] ′) coordinates.

FIG. 6 depicts the fluorescence curve of FIG. 2 that is transformed to have a baseline in (x′, [symbol] ′) coordinates.

FIG. 7 illustrates a model fit to the PCR kinetics shown in FIG. 3, where a=12.42, [symbol] =−30.92, x′₀=34.51, [symbol] ′₀=0.

FIGS. 8A and 8B depict an algorithm flow chart.

FIG. 9 shows curve from re-scaled fluorescence data in (x, [symbol] ) coordinates.

FIG. 10 shows baseline slope fitting curve that is superimposed on curve from re-scaled fluorescence data in (x, [symbol] ) coordinates.

FIG. 11 shows the corrected curve with a flat baseline in (x′, [symbol] ′) coordinates,

FIG. 12 shows the curve of FIG. 11 with the baseline minimized to almost zero fluorescence in (x′, [symbol] ′) coordinates.

FIG. 13 depicts an S-shape curve function approximation of the real-time PCR curve of FIG. 11 in (x, [symbol] ) coordinates.

FIG. 14 depicts an S-shape curve function approximation of the real-time PCR curve of FIG. 11 and the tangent line at the inflection point f′ (x′₀) in (x′, [symbol] ) coordinates.

FIG. 15 depicts an S-shape curve function approximation of the real-time PCR curve of FIG. 11 and the tangent line at the inflection point f′ (x₀) in (x, [symbol] ) coordinates.

### DETAILED DESCRIPTION

The practice of the invention employs, unless otherwise indicated, conventional molecular biological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements; Sambrook, et al., Molecular Cloning: A Laboratory Manual, 2nd Edition, Cold Spring Harbor, N.Y. (1989).

Unless defined otherwise, all technical and scientific terms used herein have the same meaning as is commonly understood by one of skill in the art. The specification also provides definitions of terms to help interpret the disclosure and claims of this application. In the event a definition is not consistent with definitions elsewhere, the definition set forth in this application will control.

### Real-Time Polymerase Chain Reactions

### Nucleic Acid Template Preparation—DNA Templates

Nucleic acid templates can be derived from humans, non-human animals, plants, bacteria, fungi, protozoa, viruses and recombinant nucleic acids such as plasmid, phage or viral vectors.

In certain embodiments, the template nucleic acid is purified from a sample which may comprise prokaryotic or eukaryotic cells, cultured cells, human or animal fluid or tissues including, but not limited to, blood, saliva, sputum, urine, feces, skin cells, hair follicles, semen, vaginal fluid, bone fragments, bone marrow, brain matter, cerebrospinal fluid, amniotic fluid, and the like. Samples may also include bacterial cells or spores (including gram+ or gram−), and viruses (including DNA-based and RNA-based). In some embodiments, the samples may be collected using swab sampling of surfaces.

Procedures for the extraction and purification of nucleic acids from samples are well known in the art (as described in Sambrook J et. al. Molecular Cloning, Cold Spring harbor Laboratory Press (1989), Ausubel et al. Short Protocols in Molecular Biology, 5th Ed. (2002) John Wiley & Sons, Inc. New York).

In addition, several commercial kits are available for the isolation of nucleic acids. Exemplary kits include, but are not limited to, Puregene DNA isolation kit (PG) (Gentra Systems, Inc., Minneapolis, Minn.), Generation Capture Column kit (GCC) (Gentra Systems, Inc.), MasterPure DNA purification kit (MP) (Epicentre Technologies, Madison, Wis.), Isoquick nucleic acid extraction kit (IQ) (Epoch Pharmaceuticals, Bothell, Wash.), NucliSens isolation kit (NS) (OrganonTeknika Corp., Durham, N.C.), QIAamp DNA Blood Mini Kit (Qiagen; Cat. No. 51104), MagNA Pure Compact Nucleic Acid Isolation Kit (Roche Applied Sciences; Cat. No. 03730964001), Stabilized Blood-to-CT™ Nucleic Acid Preparation Kit for qPCR (Invitrogen, Cat. No. 4449080) and GF-1 Viral Nucleic Acid Extraction Kit (GeneOn, Cat. No. RD05).

### Nucleic Acid Template Preparation—RNA Templates

In some embodiments, the sample is a purified RNA template (e.g., viral mRNA, total RNA, and mixtures thereof). In other embodiments, the sample may include cells from a biopsy or a lysate of cultured cells but is not limited thereto. Cells can be frozen on dry ice and stored at −70° C. prior to RNA isolation.

Procedures for the extraction and purification of RNA from samples are well known in the art. For example, total RNA can be isolated from cells using the TRIzol™ reagent (Invitrogen) extraction method. RNA quantity and quality is then determined using, for example, a Nanodrop™ spectrophotometer and an Agilent 2100 bioanalyzer (see also Peirson S N, Butler J N (2007). “RNA extraction from mammalian tissues” Methods Mol. Biol. 362: 315-27, Bird I M (2005) “Extraction of RNA from cells and tissue” Methods Mol. Med. 108: 139-48). In addition, several commercial kits are available for the isolation of RNA. Exemplary kits include, but are not limited to, RNeasy and QIAamp Viral RNA Kit (Qiagen, Valencia, Calif.) and MagMAX™ Viral RNA Isolation Kits (Ambion).

In other embodiments, RNA sequences can be obtained by T7 RNA transcription of cloned DNA sequences. An exemplary commercial kit for T7 in vitro transcription is Ambion's MEGAscript® Kit (Catalog No. 1330).

### PCR Amplification of Target Nucleic Acid Sequences

Nucleic acid amplification can be accomplished by a variety of methods, including, but not limited to, the polymerase chain reaction (PCR), nucleic acid sequence based amplification (NASBA), ligase chain reaction (LCR), strand displacement amplification (SDA) reaction, transcription mediated amplification (TMA) reaction, and rolling circle amplification (RCA). The polymerase chain reaction (PCR) is the method most commonly used to amplify specific target DNA sequences.

“Polymerase chain reaction,” or “PCR,” generally refers to a method for amplification of a desired nucleotide sequence in vitro. Generally, the PCR process consists of introducing a molar excess of two or more extendable oligonucleotide primers to a reaction mixture comprising a sample having the desired target sequence(s), where the primers are complementary to opposite strands of the double stranded target sequence. The reaction mixture is subjected to a program of thermal cycling in the presence of a DNA polymerase, resulting in the amplification of the desired target sequence flanked by the DNA primers.

The technique of PCR is described in numerous publications, including, PCR: A Practical Approach, M. J. McPherson, et al., IRL Press (1991), PCR Protocols: A Guide to Methods and Applications, by Innis, et al., Academic Press (1990), and PCR Technology: Principals and Applications for DNA Amplification, H. A. Erlich, Stockton Press (1989). PCR is also described in many U.S. patents, including U.S. Pat. Nos. 4,683,195; 4,683,202; 4,800,159; 4,965,188; 4,889,818; 5,075,216; 5,079,352; 5,104,792; 5,023,171; 5,091,310; and 5,066,584, each of which is herein incorporated by reference.

The term “sample” refers to any substance containing nucleic acid material.

Nucleic acids include, but are not limited to, synthetic DNA, plasmid DNA, genomic DNA, cDNA, hnRNA, small nuclear snRNA, mRNA, rRNA, tRNA, miRNAs, fragmented nucleic acid, nucleic acid obtained from subcellular organelles such as mitochondria or chloroplasts, and nucleic acid obtained from microorganisms or DNA or RNA viruses that may be present on or in a biological sample. Nucleic acids may be composed of a single type of sugar moiety, e.g., as in the case of RNA and DNA, or mixtures of different sugar moieties, e.g., as in the case of RNA/DNA chimeras.

A “target DNA” or “target RNA” or “target nucleic acid,” or “target nucleic acid sequence” refers to a region of a template nucleic acid that is to be analyzed.

As used herein, the term “amplification primer” or “PCR primer” or “primer” refers to an enzymatically extendable oligonucleotide that comprises a defined sequence that is designed to hybridize in an antiparallel manner with a complementary, primer-specific portion of a target nucleic acid sequence. Thus, the primer, which is generally in molar excess relative to its target polynucleotide sequence, primes template-dependent enzymatic DNA synthesis and amplification of the target sequence. A primer nucleic acid does not need to have 100% complementarity with its template subsequence for primer elongation to occur; primers with less than 100% complementarity can be sufficient for hybridization and polymerase elongation to occur provided the penultimate base at the 3′ end of the primer is able to base pair with the template nucleic acid. A PCR primer is preferably, but not necessarily, synthetic, and will generally be approximately about 10 to about 100 nucleotides in length.

Oligonucleotides may be synthesized and prepared by any suitable method (such as chemical synthesis), which is known in the art. A number of computer programs (e.g., Primer-Express) are readily available to design optimal primer sets. One of the skilled artisans would therefore easily optimize and identify primers flanking a target nucleic acid sequence of interest. For example, synthesized primers can be between 20 and 26 base pairs in length with a melting point (T_{M}) of around 55 degrees. Commercially available primers may also be used to amplify a particular target nucleic acid sequence of interest. Hence, it will be apparent to one of skill in the art that the primers and probes based on the nucleic acid information provided (or publicly available with accession numbers) can be prepared accordingly.

A “buffer” is a compound added to an amplification reaction which modifies the stability, activity, and/or longevity of one or more components of the amplification reaction by regulating the pH of the amplification reaction. The buffering agents of the invention are compatible with PCR amplification and RNase H cleavage activity.

Certain buffering agents are well known in the art and include, but are not limited to, Tris, Tricine, MOPS (3-(N-morpholino) propanesulfonic acid), and HEPES (4-(2-hydroxyethyl)-1-piperazineethanesulfonic acid). In addition, PCR buffers may generally contain up to about 70 mMKCl and about 1.5 mM or higher MgCl₂, to about 50-200 mM each of nucleotides dATP, dCTP, dGTP and dTTP.

The buffers of the invention may contain additives to optimize efficient reverse transcriptase-PCR or PCR reaction. An additive is a compound added to a composition which modifies the stability, activity, and/or longevity of one or more components of the composition. In certain embodiments, the composition is an amplification reaction composition. In certain embodiments, an additive inactivates contaminant enzymes, stabilizes protein folding, and/or decreases aggregation. Exemplary additives that may be included in an amplification reaction include, but are not limited to, bine, formamide, KCl, CaCl₂, MgOAc, MgCl₂, NaCl, NH₄OAc, NaI, Na(CO₃)₂, LiCl, MnOAc, NMP, trehalose, demethylsulfoxide (“DMSO”), glycerol, ethylene glycol, dithiothreitol (“DTT”), pyrophosphatase (including, but not limited to Thermoplasmaacidophilum inorganic pyrophosphatase (“TAP”)), bovine serum albumin (“BSA”), propylene glycol, glycinamide, CHES, Percoll™, aurintricarboxylic acid, Tween 20, Tween 21, Tween 40, Tween 60, Tween 85, Brij 30, NP-40, Triton X-100, CHAPS, CHAPSO, Mackernium, LDAO (N-dodecyl-N,N-dimethylamine-N-oxide), Zwittergent 3-10, Xwittergent 3-14, Xwittergent SB 3-16, Empigen, NDSB-20, T4G32, E. Coli SSB, RecA, nicking endonucleases, 7-deazaG, dUTP, UNG, anionic detergents, cationic detergents, non-ionic detergents, zwittergent, sterol, osmolytes, cations, and any other chemical, protein, or cofactor that may alter the efficiency of amplification. In certain embodiments, two or more additives are included in an amplification reaction. According to the invention, additives may be added to improve selectivity of primer annealing provided the additives do not interfere with the activity of RNase H.

As used herein, an “amplifying polymerase activity” refers to an enzymatic activity that catalyzes the polymerization of deoxyribonucleotides or ribonucleotides. Generally, the enzyme will initiate synthesis at the 3′ end of the primer annealed to a target nucleic acid template sequence, and will proceed toward the 5′ end of the template strand.

The amplifying nucleic acid polymerase can have one or more of the activities of a DNA-dependent DNA polymerase, a DNA-dependent RNA polymerase, a RNA-dependent DNA polymerase or a RNA dependent RNA polymerase.

A “DNA-dependent DNA polymerase activity” refers to the activity of a DNA polymerase enzyme that uses deoxyribonucleic acid (DNA) as a template for the synthesis of a complementary and anti-parallel DNA strand.

A “DNA-dependent RNA polymerase activity” refers to the activity of an RNA polymerase enzyme that uses deoxyribonucleic acid (DNA) as a template for the synthesis of an RNA strand in a process called “transcription.” (for example, Thermo T7 RNA polymerase, commercially available from Toyobo Life Science Department, Catalogue No. TRL-201)

A “RNA-dependent DNA polymerase activity” refers to the activity of a DNA polymerase enzyme that uses ribonucleic acid (RNA) as a template for the synthesis of a complementary and anti-parallel DNA strand in a process called “reverse transcription.”

A “RNA-dependent RNA polymerase activity” refers to the activity of a RNA polymerase enzyme that uses ribonucleic acid (RNA) as a template for the synthesis of a complementary RNA strand (for example, Thermus thermophilus RNA polymerase, commercially available from Cambio, Catalogue No. T90250).

### DNA Polymerase PCR Amplification

In certain embodiments, the nucleic acid polymerase is a thermostable polymerase that may have more than one of the above-specified catalytic activities.

As used herein, the term “thermostable,” as applied to an enzyme, refers to an enzyme that retains its biological activity at elevated temperatures (e.g., at 55° C. or higher), or retains its biological activity following repeated cycles of heating and cooling.

Non-limiting examples of thermostable amplifying polymerases having “DNA-dependent DNA polymerase activity” include, but are not limited to, polymerases isolated from the thermophilic bacteria Thermus aquaticus (Taq polymerase), Thermus thermophilus (Tth polymerase), Thermococcus litoralis (Tli or VENT™ polymerase), Pyrococcus furiosus (Pfu or DEEPVENT™. polymerase), Pyrococcus woosii (Pwo polymerase) and other Pyrococcus species, Bacillus stearothermophilus (Bst polymerase), Sulfolobus acidocaldarius (Sac polymerase), Thermoplasma acidophilum (Tac polymerase), Thermus rubber (Tru polymerase), Thermusbrockianus (DYNAZYME™ polymerase) (Tne polymerase), Thermotoga maritime (Tma) and other species of the Thermotoga genus (Tsp polymerase), and Methanobacterium thermoautotrophicum (Mth polymerase). The PCR reaction may contain more than one thermostable polymerase enzyme with complementary properties leading to more efficient amplification of target sequences. For example, a nucleotide polymerase with high processivity (the ability to copy large nucleotide segments) may be complemented with another nucleotide polymerase with proofreading capabilities (the ability to correct mistakes during elongation of target nucleic acid sequence), thus creating a PCR reaction that can copy a long target sequence with high fidelity. The thermostable polymerase may be used in its wild type form. Alternatively, the polymerase may be modified to contain a fragment of the enzyme or to contain a mutation that provides beneficial properties to facilitate the PCR reaction.

In one embodiment, the thermostable polymerase may be Taq DNA polymerase. Many variants of Taq polymerase with enhanced properties are known and include, but are not limited to, AmpliTaq™, AmpliTaq™, Stoffel fragment, SuperTaq™, SuperTaq™ plus, LA Taq™, LAproTaq™, and EX Taq™. In another embodiment, the thermostable polymerase is the AmpliTaq Stoffel fragment.

The technique of PCR of DNA templates is described in numerous publications, including, PCR: A Practical Approach, M. J. McPherson, et al., IRL Press (1991), PCR Protocols: A Guide to Methods and Applications, by Innis, et al., Academic Press (1990), and PCR Technology: Principals and Applications for DNA Amplification, H. A. Erlich, Stockton Press (1989). PCR is also described in many U.S. patents, including U.S. Pat. Nos. 4,683,195; 4,683,202; 4,800,159; 4,965,188; 4,889,818; 5,075,216; 5,079,352; 5,104,792; 5,023,171; 5,091,310; and 5,066,584, each of which is herein incorporated by reference.

### Reverse Transcriptase-PCR Amplification

One of the most widely used techniques to study gene expression exploits first-strand cDNA for mRNA sequence(s) as template for amplification by the PCR.

The term “reverse transcriptase activity” and “reverse transcription” refers to the enzymatic activity of a class of polymerases characterized as RNA-dependent DNA polymerases that can synthesize a DNA strand (i.e., complementary DNA, cDNA) utilizing an RNA strand as a template.

“Reverse transcriptase-PCR” of “RNA PCR” is a PCR reaction that uses RNA template and a reverse transcriptase, or an enzyme having reverse transcriptase activity, to first generate a single stranded DNA molecule prior to the multiple cycles of DNA-dependent DNA polymerase primer elongation. Multiplex PCR refers to PCR reactions that produce more than one amplified product in a single reaction, typically by the inclusion of more than two primers in a single reaction.

Exemplary reverse transcriptases include, but are not limited to, the Moloney murine leukemia virus (M-MLV) RT as described in U.S. Pat. No. 4,943,531, a mutant form of M-MLV-RT lacking RNase H activity as described in U.S. Pat. No. 5,405,776, bovine leukemia virus (BLV) RT, Rous sarcoma virus (RSV) RT, Avian Myeloblastosis Virus (AMV) RT and reverse transcriptases disclosed in U.S. Pat. No. 7,883,871.

The reverse transcriptase-PCR procedure, carried out as either an end-point or real-time assay, involves two separate molecular syntheses: (i) the synthesis of cDNA from an RNA template; and (ii) the replication of the newly synthesized cDNA through PCR amplification. To attempt to address the technical problems often associated with reverse transcriptase-PCR, a number of protocols have been developed taking into account the three basic steps of the procedure: (a) the denaturation of RNA and the hybridization of reverse primer; (b) the synthesis of cDNA; and (c) PCR amplification. In the so called “uncoupled” reverse transcriptase-PCR procedure (e.g., two step reverse transcriptase-PCR), reverse transcription is performed as an independent step using the optimal buffer condition for reverse transcriptase activity. Following cDNA synthesis, the reaction is diluted to decrease MgCl₂, and deoxyribonucleoside triphosphate (dNTP) concentrations to conditions optimal for Taq DNA Polymerase activity, and PCR is carried out according to standard conditions (see U.S. Pat. Nos. 4,683,195 and 4,683,202). By contrast, “coupled” RTPCR methods use a common buffer optimized for reverse transcriptase and Taq DNA Polymerase activities. In one version, the annealing of reverse primer is a separate step preceding the addition of enzymes, which are then added to the single reaction vessel. In another version, the reverse transcriptase activity is a component of the thermostable Tth DNA polymerase. Annealing and cDNA synthesis are performed in the presence of Mn²⁺ then PCR is carried out in the presence of Mg²⁺ after the removal of Mn²⁺ by a chelating agent. Finally, the “continuous” method (e.g., one step reverse transcriptase-PCR) integrates the three reverse transcriptase-PCR steps into a single continuous reaction that avoids the opening of the reaction tube for component or enzyme addition. Continuous reverse transcriptase-PCR has been described as a single enzyme system using the reverse transcriptase activity of thermostable Taq DNA Polymerase and Tth polymerase and as a two enzyme system using AMVRT and Taq DNA Polymerase wherein the initial 65° C. RNA denaturation step may be omitted.

In certain embodiments, one or more primers may be labeled. As used herein, “label,” “detectable label,” or “marker”, or “detectable marker”, which are interchangeably used in the specification, refers to any chemical moiety attached to a nucleotide, nucleotide polymer, or nucleic acid binding factor, wherein the attachment may be covalent or non-covalent. Preferably, the label is detectable and renders the nucleotide or nucleotide polymer detectable to the practitioner of the invention. Detectable labels include luminescent molecules, chemiluminescent molecules, fluorochromes, fluorescent quenching agents, colored molecules, radioisotopes or scintillants. Detectable labels also include any useful linker molecule (such as biotin, avidin, streptavidin, HRP, protein A, protein G, antibodies or fragments thereof, Grb2, polyhistidine, Ni2+, FLAG tags, myc tags), heavy metals, enzymes (examples include alkaline phosphatase, peroxidase and luciferase), electron donors/acceptors, acridinium esters, dyes and calorimetric substrates. It is also envisioned that a change in mass may be considered a detectable label, as is the case of surface plasmon resonance detection. The skilled artisan would readily recognize useful detectable labels that are not mentioned above, which may be employed in the operation of the present invention.

One step reverse transcriptase-PCR provides several advantages over uncoupled reverse transcriptase-PCR. One step reverse transcriptase-PCR requires less handling of the reaction mixture reagents and nucleic acid products than uncoupled reverse transcriptase-PCR (e.g., opening of the reaction tube for component or enzyme addition in between the two reaction steps), and is therefore less labor intensive, reducing the required number of person hours. One step reverse transcriptase-PCR also requires less sample, and reduces the risk of contamination. The sensitivity and specificity of one-step reverse transcriptase-PCR has proven well suited for studying expression levels of one to several genes in a given sample or the detection of pathogen RNA. Typically, this procedure has been limited to use of gene-specific primers to initiate cDNA synthesis.

The ability to measure the kinetics of a PCR reaction by on-line detection in combination with these reverse transcriptase-PCR techniques has enabled accurate and precise quantitation of RNA copy number with high sensitivity. This has become possible by detecting the reverse transcriptase-PCR product through fluorescence monitoring and measurement of PCR product during the amplification process by fluorescent dual-labeled hybridization probe technologies, such as the 5′ fluorogenic nuclease assay (“TAQMAN™”) or endonuclease assay (“CATACLEAVE™”), discussed below.

### Real-Time PCR Using a CATACLEAVE™ Probe

In other embodiments, HPV sequences are detected using Catacleave PCR. This PCR detection method employ fluorescently labeled probes that bind to the newly synthesized DNA or dyes whose fluorescence emission is increased when intercalated into double stranded DNA.

Real time detection methodologies are applicable to PCR detection of target nucleic acid sequences in genomic DNA or genomic RNA.

The probes are generally designed so that donor emission is quenched in the absence of target by fluorescence resonance energy transfer (FRET) between two chromophores. The donor chromophore, in its excited state, may transfer energy to an acceptor chromophore when the pair is in close proximity. This transfer is always non-radiative and occurs through dipole-dipole coupling. Any process that sufficiently increases the distance between the chromophores will decrease FRET efficiency such that the donor chromophore emission can be detected radiatively. Common donor chromophores include FAM, TAMRA, VIC, JOE, Cy3, Cy5, and Texas Red.) Acceptor chromophores are chosen so that their excitation spectra overlap with the emission spectrum of the donor. An example of such a pair is FAM-TAMRA. There are also non fluorescent acceptors that will quench a wide range of donors. Other examples of appropriate donor-acceptor FRET pairs will be known to those skilled in the art.

Common examples of FRET probes that can be used for real-time detection of PCR include molecular beacons (e.g., U.S. Pat. No. 5,925,517), TaqMan™ probes (e.g., U.S. Pat. Nos. 5,210,015 and 5,487,972), and CataCleave™ probes (e.g., U.S. Pat. No. 5,763,181). The molecular beacon is a single stranded oligonucleotide designed so that in the unbound state the probe forms a secondary structure where the donor and acceptor chromophores are in close proximity and donor emission is reduced. At the proper reaction temperature the beacon unfolds and specifically binds to the amplicon. Once unfolded the distance between the donor and acceptor chromophores increases such that FRET is reversed and donor emission can be monitored using specialized instrumentation. TaqMan™ and CataCleave™ technologies differ from the molecular beacon in that the FRET probes employed are cleaved such that the donor and acceptor chromophores become sufficiently separated to reverse FRET.

TaqMan™ technology employs a single stranded oligonucleotide probe that is labeled at the 5′ end with a donor chromophore and at the 3′ end with an acceptor chromophore. The DNA polymerase used for amplification must contain a 5′->3′ exonuclease activity. The TaqMan™ probe binds to one strand of the amplicon at the same time that the primer binds. As the DNA polymerase extends the primer the polymerase will eventually encounter the bound TaqMan™ probe. At this time the exonuclease activity of the polymerase will sequentially degrade the TaqMan™ probe starting at the 5′ end. As the probe is digested the mononucleotides comprising the probe are released into the reaction buffer. The donor diffuses away from the acceptor and FRET is reversed. Emission from the donor is monitored to identify probe cleavage. Because of the way TaqMan™ works a specific amplicon can be detected only once for every cycle of PCR. Extension of the primer through the TaqMan™ target site generates a double stranded product that prevents further binding of TaqMan™ probes until the amplicon is denatured in the next PCR cycle.

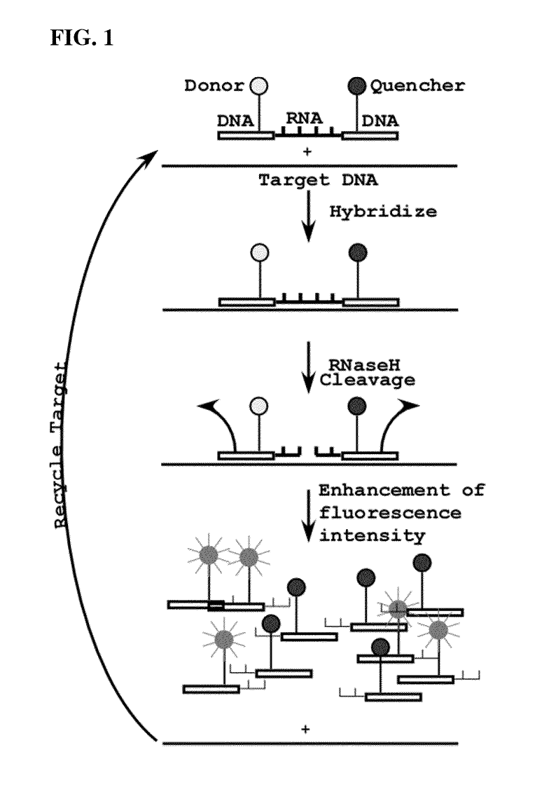

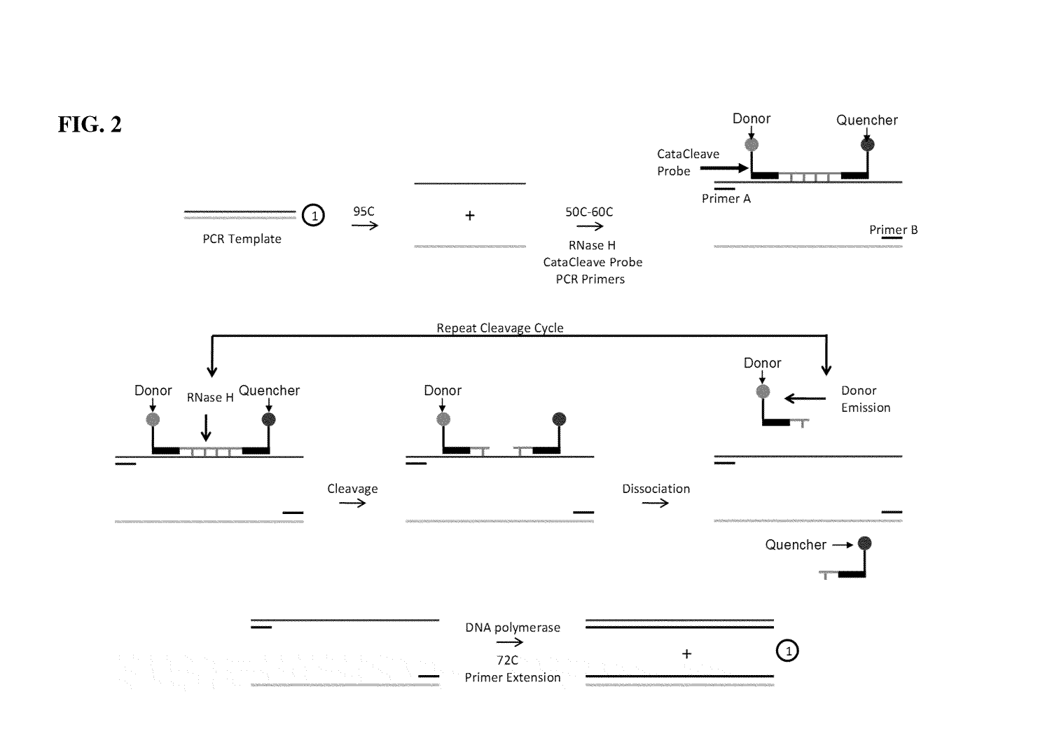

U.S. Pat. No. 5,763,181, of which content is incorporated herein by reference, describes another real-time detection method (referred to as “CataCleave™”; see FIGS. 1 and 2). CataCleave™ technology differs from TaqMan™ in that cleavage of the probe is accomplished by a second enzyme that does not have polymerase activity. The CataCleave™ probe has a sequence within the molecule which is a target of an endonuclease, such as, for example a restriction enzyme or RNase. In one example, the CataCleave™ probe has a chimeric structure where the 5′ and 3′ ends of the probe are constructed of DNA and the cleavage site contains RNA. The DNA sequence portions of the probe are labeled with a FRET pair either at the ends or internally. The PCR reaction includes a thermostable RNase H enzyme that can specifically cleave the RNA sequence portion of a RNA-DNA duplex. After cleavage, the two halves of the probe dissociate from the target amplicon at the reaction temperature and diffuse into the reaction buffer. As the donor and acceptors separate FRET is reversed in the same way as the TaqMan™ probe and donor emission can be monitored. Cleavage and dissociation regenerates a site for further CataCleave™ binding. In this way it is possible for a single amplicon to serve as a target or multiple rounds of probe cleavage until the primer is extended through the CataCleave™ probe binding site.

### Labeling of a CataCleave™ Probe

The term “probe” comprises a polynucleotide that comprises a specific portion designed to hybridize in a sequence-specific manner with a complementary region of a specific nucleic acid sequence, e.g., a target nucleic acid sequence. In one embodiment, the oligonucleotide probe is in the range of 15-60 nucleotides in length. More preferably, the oligonucleotide probe is in the range of 18-30 nucleotides in length. The precise sequence and length of an oligonucleotide probe of the invention depends in part on the nature of the target polynucleotide to which it binds. The binding location and length may be varied to achieve appropriate annealing and melting properties for a particular embodiment. Guidance for making such design choices can be found in many of the references describing TaqMan™ assays or CataCleave™, described in U.S. Pat. Nos. 5,763,181, 6,787,304, and 7,112,422, of which contents are incorporated herein by reference.

In certain embodiments, the probe is “substantially complementary” to the target nucleic acid sequence.

As used herein, the term “substantially complementary” refers to two nucleic acid strands that are sufficiently complimentary in sequence to anneal and form a stable duplex. The complementarity does not need to be perfect; there may be any number of base pair mismatches, for example, between the two nucleic acids. However, if the number of mismatches is so great that no hybridization can occur under even the least stringent hybridization conditions, the sequence is not a substantially complementary sequence. When two sequences are referred to as “substantially complementary” herein, it means that the sequences are sufficiently complementary to each other to hybridize under the selected reaction conditions. The relationship of nucleic acid complementarity and stringency of hybridization sufficient to achieve specificity is well known in the art. Two substantially complementary strands can be, for example, perfectly complementary or can contain from 1 to many mismatches so long as the hybridization conditions are sufficient to allow, for example discrimination between a pairing sequence and a non-pairing sequence. Accordingly, “substantially complementary” sequences can refer to sequences with base-pair complementarity of 100, 95, 90, 80, 75, 70, 60, 50 percent or less, or any number in between, in a double-stranded region.

As used herein, a “selected region” refers to a polynucleotide sequence of a target DNA or cDNA that anneals with the RNA sequences of a probe. In one embodiment, a “selected region” of a target DNA or cDNA can be from 1, 2, 3, 4, 5, 6, 7, 8, 9, 10, 11, 12, 13, 14, 15, 16, 17, 18, 19, 20, 21, 22, 23, 24, 25 or more nucleotides in length.

As used herein, the site-specific RNase H cleavage refers to the cleavage of the RNA moiety of the Catacleave™ probe that is entirely complimentary to and hybridizes with a target DNA sequence to form an RNA:DNA heteroduplex.

As used herein, “label” or “detectable label” of the CataCleave™ probe refers to any label comprising a fluorochrome compound that is attached to the probe by covalent or non-covalent means.

As used herein, “fluorochrome” refers to a fluorescent compound that emits light upon excitation by light of a shorter wavelength than the light that is emitted. The term “fluorescent donor” or “fluorescence donor” refers to a fluorochrome that emits light that is measured in the assays described in the present invention. More specifically, a fluorescent donor provides energy that is absorbed by a fluorescence acceptor. The term “fluorescent acceptor” or “fluorescence acceptor” refers to either a second fluorochrome or a quenching molecule that absorbs energy emitted from the fluorescence donor. The second fluorochrome absorbs the energy that is emitted from the fluorescence donor and emits light of longer wavelength than the light emitted by the fluorescence donor. The quenching molecule absorbs energy emitted by the fluorescence donor.

Any luminescent molecule, preferably a fluorochrome and/or fluorescent quencher may be used in the practice of this invention, including, for example, AlexaFluor™ 350, AlexaFluor™ 430, AlexaFluor™ 488, AlexaFluor™ 532, AlexaFluor™ 546, AlexaFluor™ 568, AlexaFluor™ 594, AlexaFluor™ 633, AlexaFluor™ 647, AlexaFluor™ 660, AlexaFluor™ 680, 7-diethylaminocoumarin-3-carboxylic acid, Fluorescein, Oregon Green 488, Oregon Green 514, Tetramethylrhodamine, Rhodamine X, Texas Red dye, QSY 7, QSY33, Dabcyl, BODIPY FL, BODIPY 630/650, BODIPY 6501665, BODIPYTMR-X, BODIPYTR-X, Dialkylaminocoumarin, Cy5.5, Cy5, Cy3.5, Cy3, DTPA(Eu³⁺)-AMCA and TTHA(Eu³⁺)AMCA.

In one embodiment, the 3′ terminal nucleotide of the oligonucleotide probe is blocked or rendered incapable of extension by a nucleic acid polymerase. Such blocking is conveniently carried out by the attachment of a reporter or quencher molecule to the terminal 3′ position of the probe.

In one embodiment, reporter molecules are fluorescent organic dyes derivatized for attachment to the terminal 3′ or terminal 5′ ends of the probe via a linking moiety. Preferably, quencher molecules are also organic dyes, which may or may not be fluorescent, depending on the embodiment of the invention. For example, in a preferred embodiment of the invention, the quencher molecule is fluorescent. Generally whether the quencher molecule is fluorescent or simply releases the transferred energy from the reporter by non-radiative decay, the absorption band of the quencher should substantially overlap the fluorescent emission band of the reporter molecule. Non-fluorescent quencher molecules that absorb energy from excited reporter molecules, but which do not release the energy radiatively, are referred to in the application as chromogenic molecules.

Exemplary reporter-quencher pairs may be selected from xanthene dyes, including fluoresceins, and rhodamine dyes. Many suitable forms of these compounds are widely available commercially with substituents on their phenyl moieties which can be used as the site for bonding or as the bonding functionality for attachment to an oligonucleotide. Another group of fluorescent compounds are the naphthylamines, having an amino group in the alpha or beta position. Included among such naphthylamino compounds are 1-dimethylaminonaphthyl-5-sulfonate, 1-anilino-8-naphthalene sulfonate and 2-p-touidinyl6-naphthalene sulfonate. Other dyes include 3-phenyl-7-isocyanatocoumarin, acridines, such as 9-isothiocyanatoacridine and acridine orange, N-(p-(2-benzoxazolyl)phenyl)maleimide, benzoxadiazoles, stilbenes, pyrenes, and the like.

In one embodiment, reporter and quencher molecules are selected from fluorescein and rhodamine dyes.

There are many linking moieties and methodologies for attaching reporter or quencher molecules to the 5′ or 3′ termini of oligonucleotides, as exemplified by the following references: Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19: 3019 (1991) (3′ sulfhydryl); Giusti et al., PCR Methods and Applications, 2: 223-227 (1993) and Fung et al., U.S. Pat. No. 4,757,141 (5′ phosphoamino group via Aminolink™. II available from Applied Biosystems, Foster City, Calif.) Stabinsky, U.S. Pat. No. 4,739,044 (3′ aminoalkylphosphoryl group); Agrawal et al., Tetrahedron Letters, 31: 1543-1546 (1990) (attachment via phosphoramidate linkages); Sproat et al., Nucleic Acids Research, 15: 4837 (1987) (5′ mercapto group); Nelson et al., Nucleic Acids Research, 17: 7187-7194 (1989) (3′ amino group); and the like.

Rhodamine and fluorescein dyes are also conveniently attached to the 5′ hydroxyl of an oligonucleotide at the conclusion of solid phase synthesis by way of dyes derivatized with a phosphoramidite moiety, e.g., Woo et al., U.S. Pat. No. 5,231,191; and Hobbs, Jr., U.S. Pat. No. 4,997,928.

### RNase H Cleavage of the CATACLEAVE™ Probe

In certain embodiments, the CatacleavePCR reaction can include a hot start RNase H activity.

Examples of RNase H enzymes, which may be employed in the embodiments, also include, but are not limited to, thermostable RNase H enzymes isolated from thermophilic organisms such as Pyrococcus furiosus, Pyrococcus horikoshi, Thermococcus litoralis or Thermus thermophilus.

Other RNase H enzymes that may be employed in the embodiments are described in, for example, U.S. Pat. No. 7,422,888 to Uemori or the published U.S. Patent Application No. 2009/0325169 to Walder, the contents of which are incorporated herein by reference.

### Baseline Slope Correction of Real-Time PCR Curves

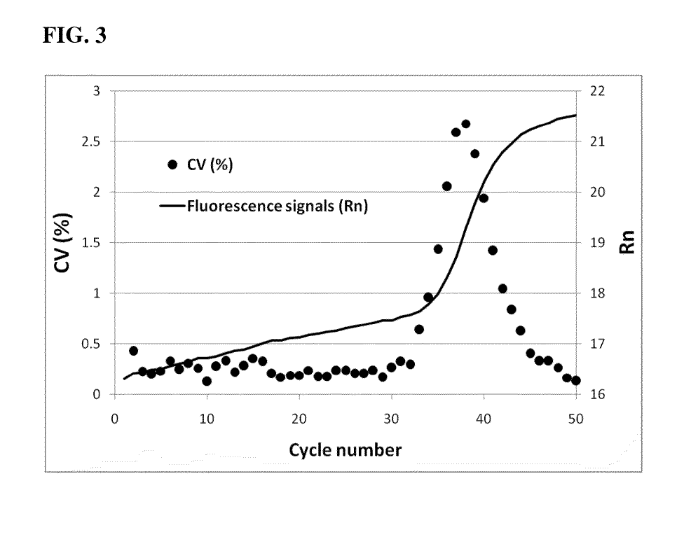

A real-time PCR curve, where fluorescence intensity is plotted against cycle number, is characterized by an S shaped curve having a baseline or the ground phase, an exponential phase and followed by a plateau (see FIG. 3).

As used herein, the sloped baseline refers to the fluorescence intensity that is detected during the initial cycles of PCR, from cycle one to about 5 or about 10 or about 15 or about 20 or about 30 depending on the starting concentration of the target nucleic acid.

The baseline phase can be distinguished from the exponential phase individually and uniquely for each sample well and/or each dye being evaluated. For each sample well and/or dye, a first plot is generated by plotting the actual signals generated by a label in the sample well as a function of cycle number. In a preferred embodiment, a second plot is generated by plotting a 3-point moving Coefficient of Variance (CV) of the actual signals as a function of cycle number by applying the equation

$${CV}_{i} = 100 \% \times \frac{{σ}_{i}}{{y}_{i}}$$

Wherein CV, is coefficient of variance of the amount of fluorescence cycles i−1, i, and i+1, and σᵢ is standard deviation of the amount of fluorescence at cycles i−1, i, and i+1, and [symbol] ᵢ is the amount of fluorescence at cycle i. The end cycle of the baseline phase or the starting cycle of the exponential phase is assigned as one of the followings: 1) a first cycle which shows a CV significantly greater than that of its previous cycle, and 2) a cycle having a CV that is equal to or greater than a specified value, e.g., 0.5. If no cycle satisfies the criteria in 1) and 2), the end cycle of the baseline phase (or the starting cycle of the exponential phase) is assigned as the total PCR cycles. The starting cycle of the baseline phase can be cycle 1, 2, 3 or any specified cycle number.

An example of changes of 3-point CV in a PCR run is illustrated in FIG. 3.

The baseline is considered to behave linearly, and it can be approximated with a linear regression by applying the equation:

y= [symbol] x+

[symbol]

where [symbol] is fluorescence at cycle x, [symbol] is slope of the baseline, and [symbol] is the intercept of the basement fitting curve with the abscissa axis.

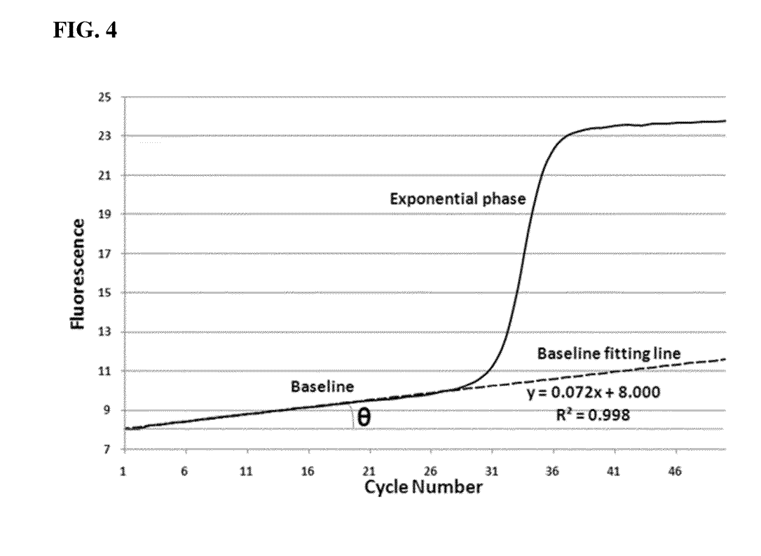

An example of a baseline fitting curve is illustrated in FIG. 4.

The angle θ corresponding to the slope of the baseline fitting (see FIG. 4) can then be corrected by converting each fluorescent data point of the first real-time PCR plot to a modified fluorescent data point (x′, [symbol] ′) by applying the equation:

$$[ \begin{matrix}{x}^{′} \\ {y}^{′}\end{matrix} ] = [ \begin{matrix}cos θ & - sin θ \\ sin θ & cos θ\end{matrix} ] [ \begin{matrix}x \\ y\end{matrix} ]$$

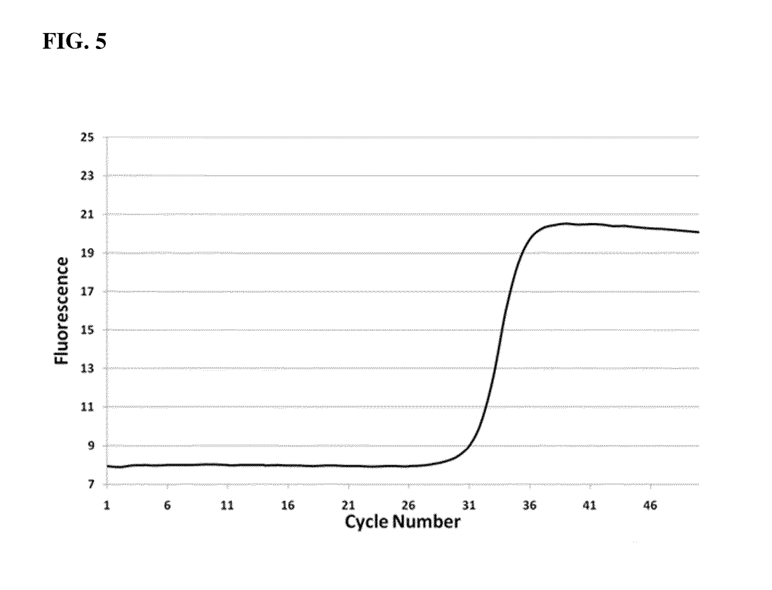

As shown in FIG. 5, plotting the modified fluorescent data points (x′, [symbol] ′) against PCR amplification cycle number results in the rotation of the real-time PCR curve counter-clockwise by an angle of θ to produce a plot with a flat baseline.

The next step entails minimizing the flat baseline to a fluorescence intensity of almost zero by subtracting the average fluorescence values of the baseline, z, from every modified fluorescence data point (x′, [symbol] ′) to approximate a baseline fitting line where “ [symbol] ′=0.”

The average fluorescence values of the baseline, y̅′, is calculated with the equation

$$\overline{{y}^{′}} = \frac{{\sum }_{i = 1}^{n} {y}_{i}^{′}}{n}$$

$$z = \sum \frac{{y}_{i}^{′}}{n}$$

where y̅′̅z is the average of the baseline fluorescence, and [symbol] ′ᵢ is fluorescence value at cycle [symbol] , and [symbol] is the total cycle number included in the baseline calculation.

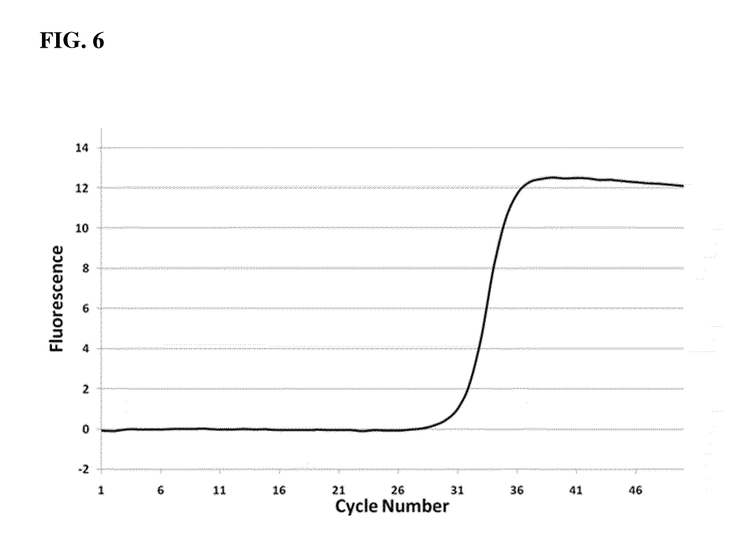

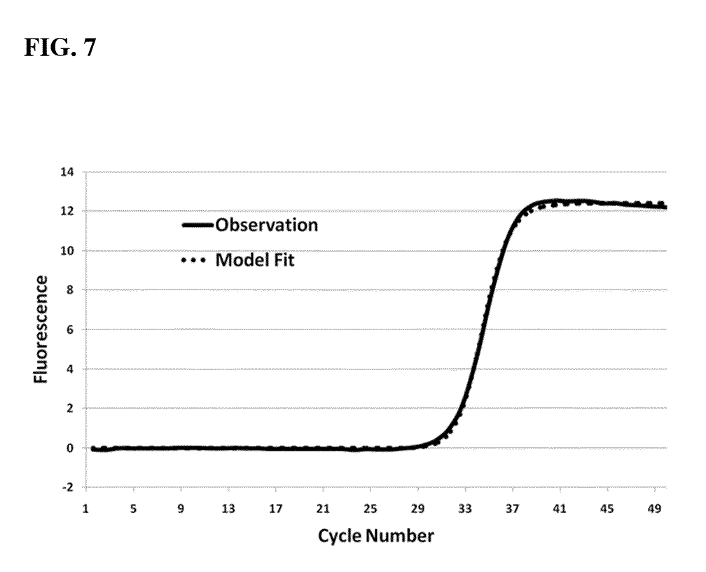

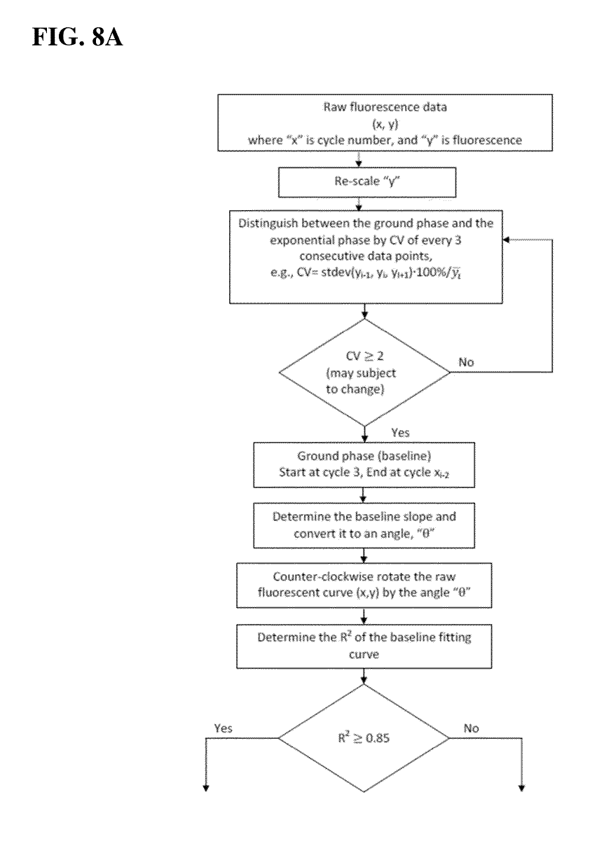

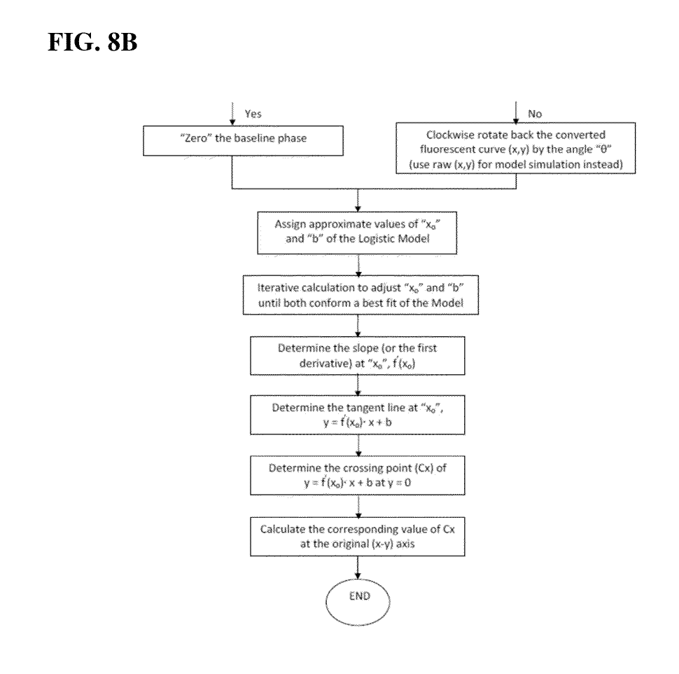

The baseline fitting line (see FIG. 6) is then determined by applying the equation:

[symbol]

″ᵢ= [symbol] ′ᵢ−

[symbol]

to each fluorescence data point of the plot with the flat base line. This transformation converts a real-time PCR curve with a high-sloped baseline into a curve having a ground phase of almost zero fluorescence.

As used herein, a “flat baseline” of a real-time PCR curve refers to a baseline that is substantially perpendicular with respect to the y axis and substantially parallel with respect to the x axis.

In certain embodiments, a “flat baseline” of a real-time PCR curve can be a baseline having a slope of about a 0 degree angle or about a 0-5 degree angle or about a 0-10 degree angle with respect to the x axis.

In other embodiments, a “flat baseline” of a real-time PCR curve can be a baseline having a slope of about a 90 degree angle or about a 85-90 degree angle or about a 80-90 degree angle with respect to the y axis.

As used herein, “almost zero fluorescence” refers to a baseline having a fluorescence value of about 0 or less than about 1% or less than about 5% of the maximal fluorescence value obtained .at the plateau phase of the real-time PCR reaction, i.e. at a cycle number equal to or greater than 40.

### Modeling of PCR Kinetics

The correction to the baseline, described above, minimizes the baseline of the real-time PCR to almost zero fluorescence. As shown in FIG. 6, this correction produces a real-time PCR plot where the baseline is flat thus facilitating the application of a mathematic model to simulate the kinetics of the PCR amplification reaction.

Mathematical models that can be applied to the corrected baseline plot include, but are not limited to, a sigmoid function, Chapman function, Richards function or Gompertz function.

As used herein, a sigmoid or S-shape curve function is a Logistic function that provides a mathematical model of the exponential increase in fluorescence intensity observed in real-time PCR amplification curves.

In one embodiment, the mathematical model can be a four parameter logistic model is applied to fit all baseline-corrected plots by applying the equation

$$f ( x ) = {y}_{0} + \frac{a}{1 + {( \frac{x}{{x}_{0}} )}^{b}}$$

where [symbol] (x) is the value of function computed (fluorescence at cycle x), [symbol] ₀ is the ground fluorescence, a is the difference between the maximal fluorescence acquired in the run and the ground fluorescence, x is the actual cycle number, x₀ is the first derivative maximum of the function, and [symbol] describes the slope of the curve at x₀, and is defined as −4· [symbol] (x₀)·x₀/a.

### Description of the Cᵢₜ Method

The ‘corrected’ crossing cycle, Cᵢₜ, corresponds to the cycle number at which a fluorescence baseline fitting curve intersects with a tangent crossing at the inflection point of a real-time PCR curve. The Cᵢₜ value is calculated by determining the intersection point between the abscissa axis and tangent of the inflection point of the logistic curve obtained by the non-linear regression of raw data. The Cᵢₜ value, like Cₜ or Cₚ, is unique to each PCR curve and dependent on the concentration of the target nucleic acid template.

The inflection point coordinate is calculated by applying the equation:

$$inflection point = [ {x}_{0} , {y}_{0} + \frac{a}{2} ]$$

The slope of the tangent line at x₀ for the four parameter logistic curve, described above, is [symbol] ′ (x₀), and the Cᵢₜ value is then calculated by applying the equation:

$${C}_{it} = - \frac{{y}_{0} + \frac{a}{2}}{{f}^{′} ( {x}_{0} )} cos θ$$

### EXAMPLES

The present invention will be described in further detail with reference to the following examples. These examples are for illustrative purposes only and are not intended to limit the scope of the invention.

### Example 1

### Mathematical Simulation of Real-Time PCR Kinetics

### Re-Scaling of Raw Data

STEP 1: Extract raw fluorescent data from either ABI or Roche instrument.
   Rₙ=y, cycle number=x.
STEP 2: If necessary raw readings of Rn (y) are re-scaled.
   Log factor=log(maximum(y))−1
The “log factor” is the rounded down to zero decimal places. The re-scale factor is 1/10^{rounded-down log factor.}

### Determination of a Baseline Phase

STEP 3: Calculate standard deviation (σᵢ) of every three consecutive y, i.e., yᵢ₋₁, yᵢ, and yᵢ₊₁. For example, σ₄ is a standard deviation of (y₃, y₄, y₅).

$${σ}_{i} = \sqrt{\frac{\sum {( y - \overline{{y}_{i}} )}^{2}}{n - 1}}$$

STEP 4: Calculate coefficient of variance (CVᵢ) of every data point.

$${CV}_{i} = 100 \% \times \frac{{σ}_{i}}{{y}_{i}}$$

STEP 5: If CVᵢ≧2, it means significant deviation of fluorescence readings from previous one, indicating a start of the exponential phase. Therefore, the cycles between 3 and (i−2) shall be the ground phase, or baseline.
   If CVᵢ<2, proceed directly to Step 10.
STEP 6: The baseline between cycles 3 and (i−2) will be fitted through linear regression. The R² value of the regression will be used to determine linearity of the baseline.

$${R}^{2} = \frac{{\sum }_{i = m}^{n} ( {x}_{i} - \overline{x} ) {( {y}_{i} - \overline{y} )}^{2}}{{\sum }_{i = m}^{n} {( {x}_{i} - \overline{x} )}^{2} {\sum }_{i = m}^{n} {( {y}_{i} - \overline{y} )}^{2 }}$$

STEP 7: If R²≧0.85, it confirms the linearity of the predicted baseline phase. Then the slope of the predicted baseline phase between cycles 3 and (i−2) is calculated. Otherwise, proceed directly to Step 10.

$$Slope = \frac{( \sum ( x - \overline{x} ) ( y - \overline{y ) )}}{\sum {( x - \overline{x} )}^{2}} )$$

### Transformation of Raw Data

STEP 8: The slope describes the steepness of the background drifting line of a CataCleave reaction. An angle, “θ”, which corresponds to the slope, is then calculated.

tan θ=slope

θ=arctan(slope)
STEP 9: Then the original fluorescent curve (x, y) is rotated counter-clock by the angle “θ”. Ideally, the baseline phase shall overlap with the new x′-axis. All (x, y) data points are then transformed to fit in the new x′-y′ axis system, i.e, (x′, y′). Now the sloped baseline becomes flat in the new x′-y′ axis system.

x′=x cos θ+y sin θ

y′=−x sin θ+y cos θ
Now the sloped baseline becomes flat (ideally it shall completely overlap with y′=0) in the new x′-y′ axis system.
STEP 10: Subtract all Rₙ (y′) values by the average of (y₃, y₄, y₅) to “zero” the baseline.

### Regression

STEP 11: Apply Logistic Model to simulate the real-time PCR kinetics.

$$f ( {x}^{′} ) = {y}_{0}^{′} + \frac{a}{1 + {( \frac{{x}^{′}}{{x}_{0}^{′}} )}^{b}}$$

Where f(x′) is fluorescence at cycle x′
y′₀ is background fluorescence, usually

$${y}_{0}^{′} = \frac{{y}_{3}^{′} + {y}_{4}^{′} + {y}_{5}^{′}}{3}$$

from observation a is maximum of y′ from observation
x′₀ is a fractional cycle number of the first derivative maximum of f(x′), i.e., inflection point.
b describes the slope of the model,

$$b = - \frac{4 {f}^{′} ( {x}_{0} ) {x}_{0}}{a}$$

STEP 12: The purpose of the Model is, by adjusting values of “x′₀” and “b”, to mimic PCR kinetics through iteration. However, to initiate the process, values of “x′₀” and “b” must be firstly assigned, as close to real numbers as possible.
STEP 13: Therefore, f′(x₀′), the first derivative of f(x₀′), can be assigned by the maximum value of

$$( \frac{{y}_{1}^{′} + {y}_{3}^{′}}{2} , \frac{{y}_{2}^{′} + {y}_{4}^{′}}{2} , \frac{{y}_{3}^{′} + {y}_{5}^{′}}{2} , \frac{{y}_{n - 2}^{′} + {y}_{n}^{′}}{2} )$$

where n is the total cycle number. Consequently, x′₀ is a cycle number corresponding to f′ (x₀′). Therefore, a value of b will be assigned accordingly.
STEP 14: With assigned values of x₀′ and b, these two parameters are repeatedly adjusted so that the Model can simulate the PCR kinetics. The iteration process continues until a minimal value of Σ(f′(x′)−y′)² is achieved.
STEP 15: Upon completion of iteration, the Logistic Regression returns final values of x₀′ and b.

### Determination of Titer-Dependent Fractional Cycle Number

STEP 16: A titer-dependent fractional cycle number is a cross point between the extension line of the baseline phase and the tangent crossing the inflection point of real-time PCR (CataCleave) fluorescent curve.
STEP 17: The tangent line crossing the inflection point (x′₀, f(x′₀)) is then defined.

$${y}^{′} = {f}^{′} ( {x}_{0}^{′} ) \cdot {x}^{′} + \frac{a}{2} + {y}_{0}^{′} - {f}^{′} ( {x}_{0}^{′} ) \cdot {x}_{0}^{′}$$

STEP 18: The fractional cycle number in the x′-y′ axis system is then calculated.

$${C}^{′} = - \frac{\frac{a}{2} + {y}_{0}^{′} - {f}^{′} ( {x}_{0}^{′} ) \cdot {x}_{0}^{′}}{{f}^{′} ( {x}_{0}^{′} )}$$

STEP 19: Then this fractional cycle number, C′, is converted back in the old x-y axis system.

$${C}_{it} = \{ \begin{matrix}\begin{matrix}{C}^{′} cos θ - {y}_{0}^{′} sin θ , & if 2 < {c}^{′} \leq 50 \\ Negative , & if {c}^{′} > 50 \\ Invalid , & if {c}^{′} \leq 2\end{matrix} \\ \begin{matrix}{C}^{′} , if no raw data transformation is ever \\ involved in caculation\end{matrix}\end{matrix}$$

### Example 2

### Real Time PCR of HBV

TABLE 2
Raw fluorescent data.

| Cycle (x) | Rₙ (y) |
|---|---|
| 1 | 1.631463 |
| 2 | 1.641601 |
| 3 | 1.643732 |
| 4 | 1.649225 |
| 5 | 1.650193 |
| 6 | 1.655965 |
| 7 | 1.660656 |
| 8 | 1.663813 |
| 9 | 1.670568 |
| 10 | 1.672028 |
| 11 | 1.675367 |
| 12 | 1.680865 |
| 13 | 1.685562 |
| 14 | 1.688483 |
| 15 | 1.69451 |
| 16 | 1.699585 |
| 17 | 1.706047 |
| 18 | 1.705579 |
| 19 | 1.711481 |
| 20 | 1.71181 |
| 21 | 1.716832 |
| 22 | 1.720155 |
| 23 | 1.723291 |
| 24 | 1.725985 |
| 25 | 1.731148 |
| 26 | 1.733912 |
| 27 | 1.737864 |
| 28 | 1.740592 |
| 29 | 1.745764 |
| 30 | 1.746029 |
| 31 | 1.753509 |
| 32 | 1.757235 |
| 33 | 1.76421 |
| 34 | 1.779132 |
| 35 | 1.79824 |
| 36 | 1.829748 |
| 37 | 1.872833 |
| 38 | 1.926677 |
| 39 | 1.976426 |
| 40 | 2.020758 |
| 41 | 2.053508 |
| 42 | 2.07854 |
| 43 | 2.097022 |
| 44 | 2.11365 |
| 45 | 2.123151 |
| 46 | 2.130527 |
| 47 | 2.137001 |
| 48 | 2.144817 |
| 49 | 2.148345 |
| 50 | 2.152005 |

Re-scale raw fluorescent data. log factor=log 2.152005−1=−0.3. Then the rescale factor is

$$\frac{1}{{10}^{- 1}} = 10.$$

Therefore, all Rₙ values in Table 1 are transformed.

TABLE 3
Re-scaled Rₙ values.

| Cycle (x) | Rn (y) |
|---|---|
| 1 | 16.31 |
| 2 | 16.42 |
| 3 | 16.44 |
| 4 | 16.49 |
| 5 | 16.50 |
| 6 | 16.56 |
| 7 | 16.61 |
| 8 | 16.64 |
| 9 | 16.71 |
| 10 | 16.72 |
| 11 | 16.75 |
| 12 | 16.81 |
| 13 | 16.86 |
| 14 | 16.88 |
| 15 | 16.95 |
| 16 | 17.00 |
| 17 | 17.06 |
| 18 | 17.06 |
| 19 | 17.11 |
| 20 | 17.12 |
| 21 | 17.17 |
| 22 | 17.20 |
| 23 | 17.23 |
| 24 | 17.26 |
| 25 | 17.31 |
| 26 | 17.34 |
| 27 | 17.38 |
| 28 | 17.41 |
| 29 | 17.46 |
| 30 | 17.46 |
| 31 | 17.54 |
| 32 | 17.57 |
| 33 | 17.64 |
| 34 | 17.79 |
| 35 | 17.98 |
| 36 | 18.30 |
| 37 | 18.73 |
| 38 | 19.27 |
| 39 | 19.76 |
| 40 | 20.21 |
| 41 | 20.54 |
| 42 | 20.79 |
| 43 | 20.97 |
| 44 | 21.14 |
| 45 | 21.23 |
| 46 | 21.31 |
| 47 | 21.37 |
| 48 | 21.45 |
| 49 | 21.48 |
| 50 | 21.52 |

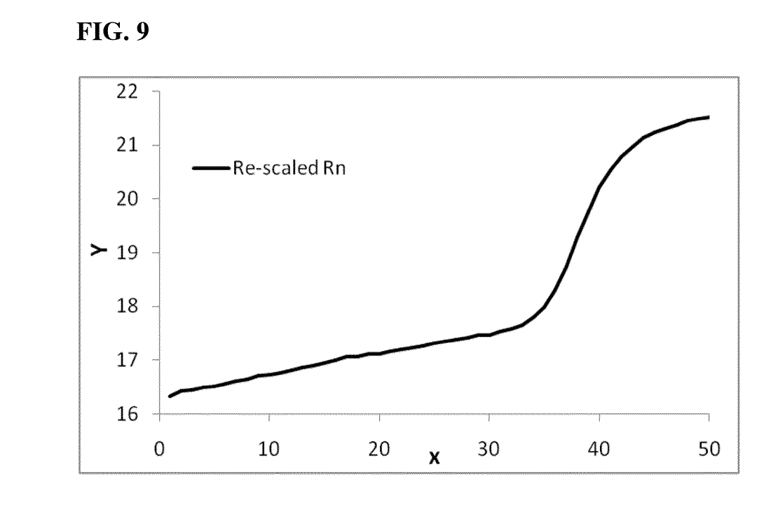

The re-scaled fluorescence data resulted in the real time PCR curve depicted in FIG. 9.

Then standard deviation and CV are also calculated from the values listed in Table 2.

TABLE 4
Standard deviation and CV of Rₙ.

| Cycle (x) | Rn (y) | Standard Deviation | CV (%) |
|---|---|---|---|
| 1 | 16.31 | | |
| 2 | 16.42 | 0.066 | 0.40 |
| 3 | 16.44 | 0.039 | 0.24 |
| 4 | 16.49 | 0.035 | 0.21 |
| 5 | 16.50 | 0.036 | 0.22 |
| 6 | 16.56 | 0.052 | 0.32 |
| 7 | 16.61 | 0.039 | 0.24 |
| 8 | 16.64 | 0.051 | 0.30 |
| 9 | 16.71 | 0.044 | 0.26 |
| 10 | 16.72 | 0.025 | 0.15 |
| 31 | 17.54 | 0.057 | 0.33 |
| 32 | 17.57 | 0.054 | 0.31 |
| 33 | 17.64 | 0.112 | 0.63 |
| 34 | 17.79 | 0.171 | 0.96 |
| 35 | 17.98 | 0.256 | 1.42 |
| 36 | 18.30 | 0.374 | 2.04 |
| 37 | 18.73 | 0.486 | 2.59 |
| 38 | 19.27 | 0.518 | 2.69 |
| 39 | 19.76 | 0.471 | 2.38 |
| 40 | 20.21 | 0.387 | 1.92 |

At cycle 36, a significant increase of CV (2.04) is observed, which means somewhere around cycle 36 sits a point separating the baseline phase and the exponential phase. Therefore, in this case the baseline phase starts from cycle 3 and ends at cycle 34.

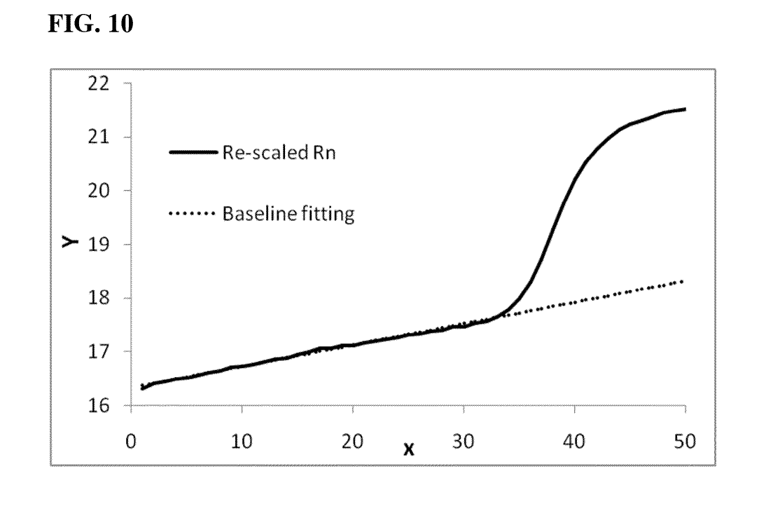

The fitting line of the baseline (between cycles 3 and 34) is, y=0.04x+16.33 with R²=0.995. “R²>0.9” confirms the validity of the baseline prediction (see FIG. 10).

Then cos θ and sin θ can be derived from the slope (0.04) of the fitting line. In this case, cos θ=0.999, sin θ=0.040. Then all data points (x,y) in the x-y axis are transformed to (x′, y′) in the new x′-y′ axis system.

For example, a data point (3, 16.44) will be converted (3.65, 16.30).

x′=3.65=3×0.999+16.44×0.040

y′=16.30=−3×0.040+16.44×0.999

TABLE 5
Transformation from (x, y) to (x′, y′).

| | |
|---|---|
| x-y axis system | x′-y′ axis system |

| Cycle (x) | Rn (y) | Cycle (x′) | Rn (y′) |
|---|---|---|---|
| 1 | 16.31 | 1.65 | 16.26 |
| 2 | 16.42 | 2.65 | 16.32 |
| 3 | 16.44 | 3.65 | 16.30 |
| 4 | 16.49 | 4.65 | 16.32 |
| 5 | 16.50 | 5.65 | 16.29 |
| 6 | 16.56 | 6.65 | 16.31 |
| 7 | 16.61 | 7.65 | 16.32 |
| 8 | 16.64 | 8.66 | 16.31 |
| 9 | 16.71 | 9.66 | 16.33 |
| 10 | 16.72 | 10.66 | 16.31 |
| 31 | 17.54 | 31.67 | 16.29 |
| 32 | 17.57 | 32.67 | 16.29 |
| 33 | 17.64 | 33.68 | 16.32 |
| 34 | 17.79 | 34.68 | 16.42 |
| 35 | 17.98 | 35.69 | 16.58 |
| 36 | 18.30 | 36.70 | 16.85 |
| 37 | 18.73 | 37.72 | 17.24 |
| 38 | 19.27 | 38.74 | 17.74 |
| 39 | 19.76 | 39.76 | 18.20 |
| 40 | 20.21 | 40.77 | 18.60 |

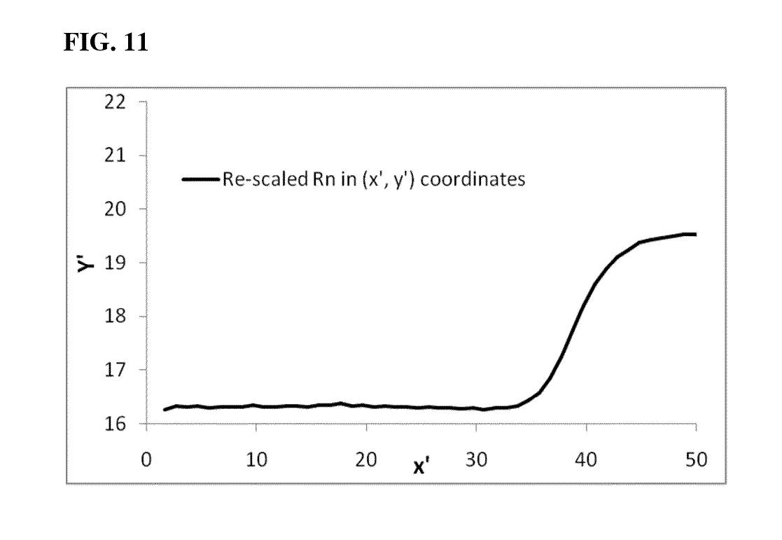

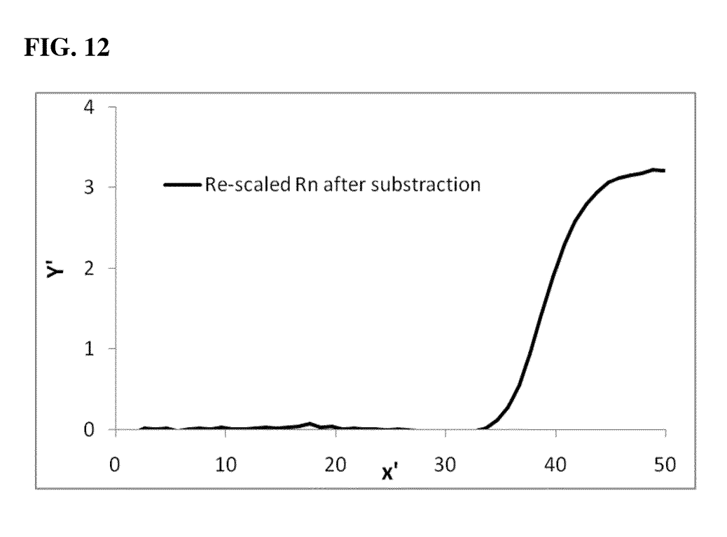

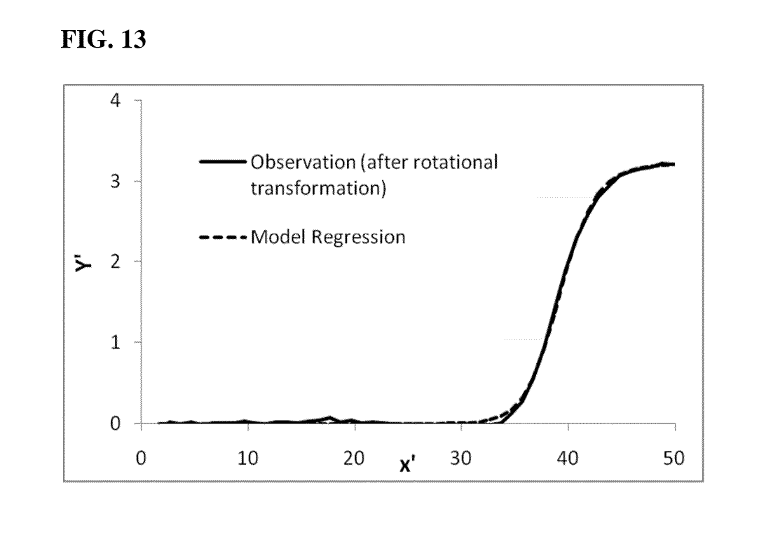

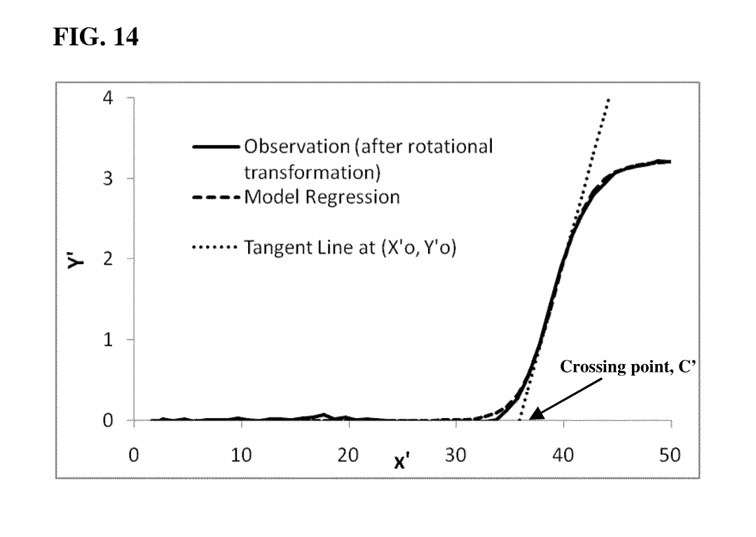

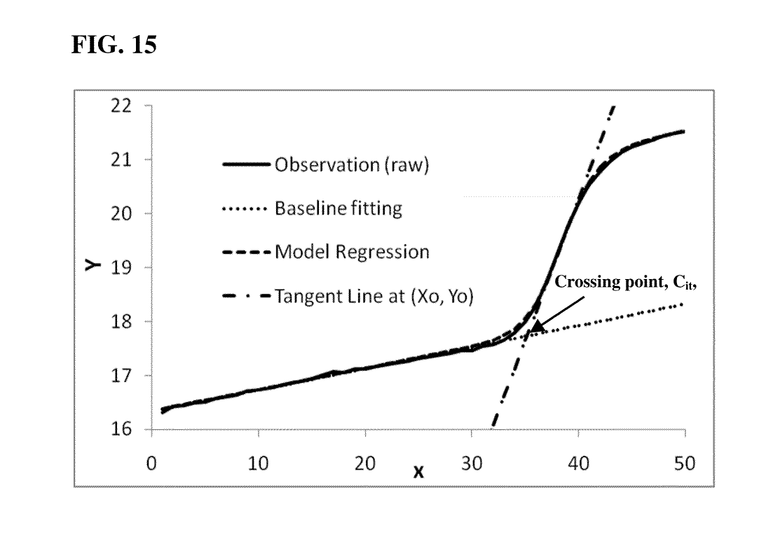

FIG. 11 shows the corrected curve with a flat baseline. All Rₙ (y′) values are then subtracted by the average of (y₃, y₄, y₅) to “zero” the baseline (see FIG. 12).

The Logistic Model is then applied to simulate the PCR kinetics. “xₒ” of first derivative maximum is first assigned, and then “b”. The approximation of the first derivative is calculated.

TABLE 6
Estimation of 1^{st} derivative.

| | | 1^{st} derivative |
|---|---|---|
| Cycle (x′) | Rn (y′) | (approximation) |
| 1.65 | −0.04 | |
| 2.65 | 0.02 | 0.022 |
| 3.65 | 0.00 | −0.002 |
| 4.65 | 0.02 | −0.007 |
| 5.65 | −0.02 | −0.006 |
| 6.65 | 0.00 | 0.012 |
| 7.65 | 0.01 | −0.001 |
| 8.66 | 0.00 | 0.010 |
| 9.66 | 0.03 | 0.001 |
| 10.66 | 0.00 | −0.016 |
| 31.67 | −0.02 | 0.016 |
| 32.67 | −0.02 | 0.014 |
| 33.68 | 0.01 | 0.070 |
| 34.68 | 0.12 | 0.130 |
| 35.69 | 0.27 | 0.213 |
| 36.70 | 0.55 | 0.333 |
| 37.72 | 0.94 | 0.444 |
| 38.74 | 1.44 | 0.478 |
| 39.76 | 1.89 | 0.430 |
| 40.77 | 2.30 | 0.345 |

The 1^{st} derivative maximum is 0.478 at cycle 38.74, then

x₀=38.74.

a=3.22 when x′=48.82.

y′₀=0

Then b=−4×0.478×38.74/3.22=−23.01

Using x′₀=38.74 and b=−23.01 as the first try to start the iteration process of the Logistic Model until Σ(f′(x′)−y′)² reaches a minimal value. In this case, when Σ(f′(x′)−y′)²=0.05, the iteration stops.

When Σ(f′(x′)−y′)²=0.05, the Model returns final values for the two parameters, x′₀=39.22 and b=−23.35.

Therefore, the first derivative maximum, i.e., the slope of the tangent line at the inflection point is f′ (x′₀), which is −3.22×(−23.35)/(4×39.22)=0.48. And the intercept is (3.22/2)+0−0.48*39.22=−17.17.

Then C′=−17.17/0.48=35.86. Since 2<C′<50, then

Cᵢₜ=35.86×0.999−0×0.040=35.82.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material set forth herein is only incorporated to the extent that no conflict arises between that incorporated material and the present disclosure material.

1. A method of correcting a real-time PCR curve, comprising:
detecting an optical signal emitted during a real-time PCR amplification of a target nucleic acid;
plotting the intensity of the optical signal as a function of cycle number to obtain a first real-time PCR plot having a baseline phase and an exponential phase;
distinguishing the end cycle of the baseline phase from the starting cycle of the exponential phase;
generating a baseline slope fitting curve of the first plot;
converting the first plot to a second plot with a flat baseline;
transforming the second plot into a third plot having a baseline that is minimized to almost zero fluorescence, and
determining the concentration of the target nucleic acid in the sample.

2. The method of correcting a real-time PCR curve according to claim 1, wherein the end cycle of the baseline phase or the starting cycle of the exponential phase is determined by plotting a 3-point moving Coefficient of Variance (CV) of the actual signals as a function of cycle number by applying the equation
$${CV}_{i} = 100 \% \times \frac{{σ}_{i}}{{y}_{i}}$$
wherein CVᵢ is a coefficient of variance of the amount of fluorescence cycles i−1, i, and i+1, and
σᵢ is standard deviation of the amount of fluorescence at cycles i−1, i, and i+1, and [symbol]
ᵢ is the amount of fluorescence at cycle i.

3. The method of correcting a real-time PCR curve according to claim 2, wherein the end cycle of the baseline phase or the starting cycle of the exponential phase is assigned by
a cycle which first shows a Coefficient of Variance (CV) significantly greater than that of its previous cycle, or
a cycle having a Coefficient of Variance (CV) from about 0.1 to about 10.

4. The method of correcting a real-time PCR curve according to claim 3,
wherein if the end cycle of the baseline phase or the starting cycle of the exponential phase is not assigned by the cycle which first shows a CV significantly greater than that of its previous cycle or by a cycle having a CV from about 0.1 to about 10,
the end cycle of the baseline phase or the starting cycle of the exponential phase is assigned by the total number of PCR cycles.

5. The method of correcting a real-time PCR curve according to claim 1, wherein the starting cycle of the baseline phase can be any cycle number.

6. The method of correcting a real-time PCR curve according to claim 1, wherein the baseline slope fitting curve is a linear regression that is determined by applying the equation
y= [symbol] x+ [symbol]
to each fluorescence data point of the first plot's baseline,
wherein [symbol] is the amount of fluorescence at cycle x, [symbol] is slope of the baseline fitting curve, and [symbol] is the value at the intercept of the curve with the abscissa axis.

7. The method of correcting a real-time PCR curve according to claim 6, wherein the linearity of the baseline fitting curve can be determined by the square of the Pearson Coefficient (R²) by applying the equation:
$${R}^{2} = \frac{{\sum }_{i = m}^{n} ( {x}_{i} - \overline{x} ) {( {y}_{i} - \overline{y} )}^{2}}{{\sum }_{i = m}^{n} {( {x}_{i} - \overline{x} )}^{2} {\sum }_{i = m}^{n} {( {y}_{i} - \overline{y} )}^{2}}$$

8. The method of correcting a real-time PCR curve according to claim 6, wherein cycle x starts at cycle 1 and increases incrementally until the beginning of the first real-time PCR plot's exponential phase.

9. The method of correcting a real-time PCR curve according to claim 1, wherein the second plot with a flat baseline is produced by converting each fluorescent data point (x, [symbol] ) of the first real-time PCR plot into a modified fluorescent data point (x′, [symbol] ′) by applying the equation:
$$[ \begin{matrix}{x}^{′} \\ {y}^{′}\end{matrix} ] = [ \begin{matrix}cos θ & - sin θ \\ sin θ & cos θ\end{matrix} ] [ \begin{matrix}x \\ y\end{matrix} ]$$
to each fluorescent data point of the first real-time PCR plot,
wherein θ is the angle corresponding to the slope of the baseline fitting curve.

10. The method of correcting a real-time PCR curve according to claim 1, wherein the third plot is generated with fluorescent data points that are corrected to minimize baseline fluorescence, each corrected fluorescent data point, [symbol] ″ᵢ, at cycle xᵢ, being determined by subtracting the average fluorescence value of baseline, y̅′̅, from each modified fluorescence value ( [symbol] ′ᵢ) by applying the equation: [symbol]
″ᵢ= [symbol] ′ᵢ− [symbol]
wherein the average value of the baseline, y̅′̅, is calculated by applying the equation:
$$\overline{{y}^{′}} = \frac{{\sum }_{i = 1}^{n} {y}_{i}^{′}}{n}$$
wherein [symbol] ′ᵢ is the value of the modified fluorescent data point at cycle xᵢ, and xₙ is the total cycle number included in the baseline calculation.

11. The method of correcting a real-time PCR curve according to claim 1,
wherein said real-time PCR amplification comprises the steps of:
providing a sample to be tested for the presence of a target DNA sequence;
providing a pair of forward and reverse amplification primers, wherein the forward amplification primer anneals to the 5′ end of the target nucleic acid sequence and the reverse amplification primer anneals to the 3′ end of the target nucleic acid sequence;
providing a probe comprising a detectable label and DNA and RNA nucleic acid sequences, wherein the probe's RNA nucleic acid sequences are entirely complementary to a selected region of the target DNA and the probe's DNA nucleic acid sequences are substantially complementary to sequences adjacent to the selected region of the target DNA sequence, and
amplifying a PCR fragment between the forward and reverse amplification primers in the presence of the target DNA sequence, an amplifying polymerase and an amplification buffer comprising a thermostable RNase H activity under conditions where the RNA sequences within the probe can form a RNA:DNA heteroduplex with complimentary sequences in the PCR fragment,
wherein cleavage of RNA sequences within the RNA:DNA heteroduplex by the RNase H activity results in the emission of the optical signal from the label on the probe.

12. The method of correcting a real-time PCR curve with a sloped baseline according to claim 11, wherein the detectable label on the probe is a fluorescent label.

13. The method of correcting a real-time PCR curve with a sloped baseline according to claim 12, wherein the fluorescent label is a FRET pair.

14. The method of correcting a real-time PCR curve according to claim 1, wherein the target nucleic acid sequence is a cDNA of a target RNA sequence.

15. A method of determining an S-shape curve function that fits a baseline slope corrected real-time PCR curve, comprising:
detecting an optical signal emitted during a real-time PCR amplification of a target nucleic acid;
plotting the intensity of the optical signal as a function of cycle number to obtain a first real-time PCR plot having a baseline phase and an exponential phase;
distinguishing the end cycle of the baseline phase from the starting cycle of the exponential phase;
transforming the first real-time PCR plot into a baseline slope corrected second plot; and
correlating an S-shape curve function from the baseline slope corrected second plot.

16. The method of determining an S-shape curve function that fits a baseline slope corrected real-time PCR curve according to claim 15, wherein correlating the S-shape curve function from the baseline slope corrected second plot comprises the steps of applying the equation
$$f ( x ) = {y}_{0} + \frac{a}{1 + {( \frac{x}{{x}_{0}} )}^{b}}$$
to each fluorescent data point of the baseline corrected second plot,
wherein [symbol] (x) is the value of function computed for a fluorescence data point at cycle x, [symbol] ₀ is the ground fluorescence, a, is the difference between the maximal fluorescence acquired in the run and the ground fluorescence, x is the actual cycle number, x₀ is the first derivative maximum of the function, and [symbol] describes the slope of the curve at x₀, and is defined by the equation −4· [symbol] (x₀)·x₀/a.

17. The method of determining an S-shape curve function that fits a baseline slope corrected real-time PCR curve according to claim 15, wherein transforming the first real-time PCR plot into a baseline slope corrected second plot comprises the steps of:
generating a baseline slope fitting curve of the first real-time PCR plot having a baseline phase and an exponential phase;
distinguishing the end cycle of the baseline phase from the starting cycle of the exponential phase;
converting the first real-time PCR plot to a second plot with a flat baseline; and
transforming the second plot into a third plot having a baseline that is minimized to almost zero fluorescence.

18. The method of determining an S-shape curve function that fits a baseline slope corrected real-time PCR curve according to claim 15, wherein the second plot with a flat baseline is produced by converting each fluorescent data point (x, [symbol] ) into a modified fluorescent data point (x′, [symbol] ′) by applying the equation
$$[ \begin{matrix}{x}^{′} \\ {y}^{′}\end{matrix} ] = [ \begin{matrix}cos θ & - sin θ \\ sin θ & cos θ\end{matrix} ] [ \begin{matrix}x \\ y\end{matrix} ]$$
to each fluorescent data point of the first real-time PCR plot,
wherein θ is the angle corresponding to the slope of the baseline fitting curve.

19. The method of determining an S-shape curve function that fits a baseline slope corrected real-time PCR curve according to claim 18, wherein the third plot is generated with fluorescent data points that are corrected to minimize baseline fluorescence, each corrected fluorescent data point, [symbol] ″ᵢ, at cycle [symbol] , being determined by subtracting the average fluorescence value of baseline, y̅′̅z, from each modified fluorescence value ( [symbol] ′ᵢ) by applying the equation: [symbol]
″ᵢ= [symbol] ′ᵢ− [symbol]
wherein the average value of the baseline, z, is calculated by applying the equation:
$$\overline{{y}^{′}} = \frac{{\sum }_{i = 1}^{n} {y}_{i}^{′}}{n}$$
wherein [symbol] ′ᵢ is the value of the modified fluorescent data point at cycle xᵢ, and xₙ is the total cycle number included in the baseline calculation.

20. A method of determining the threshold cycle, Cᵢₜ, comprising:
detecting an optical signal emitted during a real-time PCR amplification of a target nucleic acid;
plotting the intensity of the optical signal as a function of cycle number to obtain a first real-time PCR plot having a baseline phase and an exponential phase;
distinguishing the end cycle of the baseline phase from the starting cycle of the exponential phase;
transforming the first real-time PCR plot into a baseline slope corrected plot having a baseline that is minimized to almost zero fluorescence,
correlating an S-shape curve function from the baseline slope corrected plot; and
determining a Cᵢₜ value at the intersection point between the abscissa axis and tangent of the inflection point of the S-shape curve function;
wherein the Cit value is a measurement of the concentration of the nucleic acid in the sample.

21. The method of determining the threshold cycle, Cᵢₜ, according to claim 20, wherein the inflection point coordinate is calculated by applying the equation:
$$inflection point = [ {x}_{0} , {y}_{0} + \frac{a}{2} ]$$
wherein the slope of the tangent line at x₀ is [symbol] ′ (x₀), and the Cᵢₜ value is calculated with the equation:
$${C}_{it} = - \frac{{y}_{0} + \frac{a}{2}}{{f}^{′} ( {x}_{0} )} cos θ .$$

22. The method of determining the threshold cycle, Cᵢₜ, according to claim 20, wherein transforming the first real-time PCR plot into a second plot having a baseline that is minimized to almost zero fluorescence comprises the steps of:
producing the second plot with a flat baseline by converting each fluorescent data point (x, [symbol] ) of the real-time PCR plot into a modified fluorescent data point (x′, [symbol] ′) by applying the equation:
$$[ \begin{matrix}{x}^{′} \\ {y}^{′}\end{matrix} ] = [ \begin{matrix}cos θ & - sin θ \\ sin θ & cos θ\end{matrix} ] [ \begin{matrix}x \\ y\end{matrix} ]$$
wherein θ is the angle corresponding to the slope of the baseline fitting curve; and
determining a corrected fluorescent data point, [symbol] ″ᵢ, at cycle xᵢ, from the baseline fitting curve by subtracting the average fluorescence value of the baseline, y̅′̅z, from each modified fluorescence value ( [symbol] ′ᵢ) by applying the equation: [symbol]
″ᵢ= [symbol] ′ᵢ− [symbol]
wherein the average value of the baseline, y̅′̅z, is calculated by applying the equation:
$$\overline{{y}^{′}} = \frac{{\sum }_{i = 1}^{n} {y}_{i}^{′}}{n}$$
wherein [symbol] ′ᵢ is the value of the modified fluorescent data point at cycle xᵢ, and xₙ is the total cycle number included in the baseline calculation.

23. The method of determining the threshold cycle, Cᵢₜ, according to claim 20, wherein correlating the S-shape curve function from the baseline slope corrected plot comprises the steps of applying the equation
$$f ( x ) = {y}_{0} + \frac{a}{1 + {( \frac{x}{{x}_{0}} )}^{b}}$$
wherein [symbol] (x) is the value of the function computed for a fluorescence data point at cycle x, [symbol] ₀ is the ground fluorescence, a, is the difference between the maximal fluorescence acquired in the run and the ground fluorescence, x is the actual cycle number, x₀ is the first derivative maximum of the function, and [symbol] describes the slope of the curve at x₀, and is defined as −4· [symbol] (x₀)·x₀/a.

24. The method of determining the threshold cycle, Cᵢₜ, according to claim 20, wherein said real-time PCR amplification comprises the steps of:
providing a sample to be tested for the presence of a target DNA sequence;
providing a pair of forward and reverse amplification primers, wherein the forward amplification primer anneals to the 5′ end of the target nucleic acid sequence and the reverse amplification primer anneals to the 3′ end of the target nucleic acid sequence;
providing a probe comprising a detectable label and DNA and RNA nucleic acid sequences, wherein the probe's RNA nucleic acid sequences are entirely complementary to a selected region of the target DNA and the probe's DNA nucleic acid sequences are substantially complementary to sequences adjacent to the selected region of the target DNA sequence, and
amplifying a PCR fragment between the forward and reverse amplification primers in the presence of the target DNA sequence, an amplifying polymerase and an amplification buffer comprising a thermostable RNase H activity under conditions where the RNA sequences within the probe can form a RNA:DNA heteroduplex with complimentary sequences in the PCR fragment,
wherein cleavage of RNA sequences within the RNA:DNA heteroduplex by the RNase H activity results in the emission of the optical signal from the label on the probe.

25. The method of determining the threshold cycle, Cᵢₜ, according to claim 24, wherein the label is a FRET pair.
